(19) United States
(12) Patent Application Publication
(10) Pub. No.: US 2011/0239716 A1
(43) Pub. Date: Oct. 6, 2011

(54) USEFUL PRODUCT PRODUCING APPARATUS, USEFUL PRODUCT PRODUCED BY SAME AND METHOD FOR PRODUCING USEFUL PRODUCT

(76) Inventor: Koji Miyanouchi, Ishikawa (JP)

(21) Appl. No.: 13/088,923

(22) Filed: Apr. 18, 2011

Related U.S. Application Data
(63) Continuation of application No. PCT/JP2008/068862, filed on Oct. 17, 2008.

(51) Int. Cl. C05F 11/08; C12M 1/107; C10L 1/30; A23K 1/00

(52) U.S. Cl. 71/8; 435/300.1; 435/290.1; 44/307; 426/7

(57) ABSTRACT

It is an object of the present invention to provide a compact processing apparatus which can produce useful products efficiently without producing bad odor. There is provided a useful product producing apparatus that is a decomposing apparatus of organic wastes, the decomposing apparatus comprising: a fermentation device including a plurality of processing tanks for decomposing the organic wastes; an air supply device that forcibly supplies air into the plurality of processing tanks; and a deodorizing device that deodorizes gases evacuated from each processing tank, in which useful products are produced from the organic wastes in the fermentation device by a thermoduric bacteria flora, and the gases are deodorized by passing through an acid pickling tank, a rinsing tank, and a microbial deodorizing tank, successively, the acid pickling tank, the rinsing tank, and the microbial deodorizing tank configuring the deodorizing device.

### RELATED APPLICATION

This is a continuation application of the international patent application No. PCT/JP2008/068862 filed with application date: Oct. 18, 2008. The present application is based on the PCT application, and the disclosure of which is hereby incorporated by reference herein its entirety.

### BACKGROUND OF THE INVENTION

The present invention relates to a producing apparatus that converts mixed wastes produced through human livelihoods into a useful product and a method for producing the same. More specifically, the present invention relates to an apparatus and a method which can convert organic wastes into a useful product, such as a fertilizer, a feeding stuff, or a fuel, using microorganisms.

### BACKGROUND ART

Since ancient times, mixed wastes produced through human livelihoods are used efficiently through a cycle such that mixed wastes are left at a waste collection site provided in order to maintain a hygienic environment, decomposed by microorganisms present in nature, and returned to the earth. Moreover, there is a tradition such that grasses, woods, etc., are collected at a site in order to produce composts, and such composts are dispersed, or mowed feed crops are fermented in order to produce feeding stuffs (silage).

However, the more the population increases, the more the amount of the above-explained wastes increases, and the kinds thereof become varied. When the capacity of microorganisms present in nature becomes unable to cope with the amount or quality of the wastes, it results in environmental pollution, etc.

Conversely, a fermentation reaction by bacteria present in nature and the production process of composts often produce bad odor

Organic wastes among wastes can be converted into feeding stuffs, fertilizer, and other useful products by appropriately decomposing such organic wastes using aerobic bacteria present in nature, so that effective utilization thereof is enabled through such a cycle. Conversely, when the amount of wastes ejected through human livelihoods is taken into consideration, a capacity equal to or larger than a certain level is necessary, but in order to ensure such efficient process, it is necessary to create an environment appropriate for the growth of aerobic bacteria and to maintain such an environment.

In order to overcome such a circumstance, various waste processing apparatuses have been proposed so far.
Patent Literature 1: JP S58-35957A
Patent Literature 2: JP H06-57106A
Patent Literature 3: JP H08-13714A
Patent Literature 4: JP 1-108-25827A
Patent Literature 5: Japan Patent No. 3600932

### SUMMARY OF THE INVENTION

JP S58-35957A (hereinafter, referred to as a first prior art) among the technologies proposed so far discloses a rapid fermentation composting apparatus which has axial-flow rotating blades provided in a primary fermentation tank, and at least four blades are overlapped so as to form a space S at mutually adjoining locations in the bottom. Each blade is formed with a tiny opening, and such tiny openings are communicated with an air pipe having a spray orifice.

This apparatus has a shorter tune for fermentation than planar composting apparatuses, and can use microorganisms more efficiently.

This rapid fermentation composting apparatus is, however, for fecal wastes of farm animals, and is not always appropriate for the fermentation process of various organic wastes having different sizes, shapes, and origins, etc. Moreover, there is no disclosure about deodorizing.

JP H06-57106A (hereinafter, referred to as a second prior art) discloses a technology of producing fermented feeding stuffs derived from fish bodies. According to the second prior art, air is blown from the bottom of a fermentation tank and fish bodies are buried into rotten green things which generate fermentation heat equal to or higher than 70° C. in order to cause the fish bodies to be fermented while maintaining the temperature.

In comparison with conventional technique through extraction of oils by boiling the fish bodies with water, compression dewatering, break-up after drying by hot air, and productization, the above-explained technique does not need a large amount of heat sources and a treatment of drain water.

According to the above-explained technique, however, it is necessary to pick out the fish bodies buried in the rotten green things after a predetermined period has elapsed, and the rotten green things are not stirred so that the fermentation condition in the apparatus is non-uniform. Moreover, there is no disclosure about deodorizing like the first prior art.

JP H08-13714A (hereinafter, referred to as a third prior art) discloses a technology of adding microorganisms belonging to heterotrophic group to the shells of shellfish in order to execute a fermentation process under an aerobic condition, and of producing organic fermented fertilizer.

The third prior art can inexpensively produce a large amount of organic fermented fertilizers which have remarkable effects in epidemic prevention of green things, aggregation of earth medium, and water retentivity thereof, using microorganisms belonging to heterotrophic group relating to decomposing of chitin. However, there is no disclosure about deodorizing.

According to a fertilizer producing apparatus of this third prior art, the microorganisms used belong to heterotrophic group which are appropriate for decomposing of chitin, but the possibility of fermentation of other wastes is unknown. Moreover, a mixing device and a fermentation device are separated, so that there is a difficulty in uniform fermentation in the fermentation device.

JP H08-25827A (hereinafter, referred to as a fourth prior art) discloses a producing technique of fermented feeding stuffs derived from fish bodies. According to the fourth prior art, air is blown from the bottom of a fermentation tank and fish bodies are buried into rotten green things which generate fermentation heat equal to or higher than 70° C. in order to cause the fish bodies to be fermented while maintaining the temperature.

This technique can inexpensively produce fermented fertilizers containing little crude fat and a large amount of amino acid.

According to this technique, however, like the second prior art, it is necessary to pick out the fish bodies buried in the rotten green things after a predetermined period has elapsed, and the rotten green things are not stirred so that the fermentation condition in the apparatus is non-uniform. Moreover, there is no disclosure about deodorizing.

Japan Patent No. 3600932 (hereinafter, referred to as a fifth prior art) discloses microorganisms with a deodorizing action. The fifth prior art also discloses a technique of using peat moss to which such microorganisms are immobilized and which will be used as an filling material, and of deodorizing odor produced through the processing of organic wastes by using such filling material. However, there is no disclosure about fermentation of such organic wastes.

According to the above-explained conventional waste fermentation apparatuses, even if loaded wastes form a layer at the time of loading thereof, when processed objects are to be ejected, the processed objects near the ejection port can be ejected but the processed objects apart from the ejection port cannot be ejected.

Hence, there is a strong request of a compact processing apparatus which can let loaded wastes fermented for a certain period while maintaining the layer formed by the loaded wastes as it is, is capable of directly eject such a layer from a fermentation tank, and includes a deodorizing device with a high deodorizing effect.

The inventor of the present invention keenly kept researching, and successfully developed a compact process apparatus which allows wastes to be fermented for a certain period while maintaining layers formed by loaded wastes as those are, is capable of conveying those layers from a fermentation tank as those are, and has a deodorizing device with a superior deodorizing effect.

That is, the present invention provides a useful product producing apparatus which is a decomposing apparatus of organic wastes and includes: a fermentation device including a primary fermentation tank and a secondary fermentation tank and for decomposing the organic wastes; an air supply device that forcibly supplies air to each of the primary fermentation tank and the secondary fermentation tank; a deodorizing device that deodorizes gases evacuated from the primary fermentation tank and the secondary fermentation tank; and a control device that controls the air supply device in order to control a fermentation process in the fermentation device, in which the useful product producing apparatus further comprises first convey means for conveying, to a second loading opening provided in an upper portion of the secondary fermentation tank, primary processed materials that are ejected from a first ejecting opening provided at a bottom portion of the primary fermentation tank after the organic wastes loaded in a first loading opening provided in an upper portion of the primary fermentation tank are processed in the primary fermentation tank, the primary fermentation tank includes a first ejecting device which ejects a first lowermost layer from the first ejecting opening as the primary processed materials and which moves first stacked materials having the first lowermost layer eliminated therefrom downwardly in a horizontal condition after a process time of the first lowermost layer that is the lowermost layer in the first stacked materials has elapsed by a predetermined first process time in the primary fermentation tank, the first stacked materials including a plurality of substantially horizontal layers stacked together, the plurality of layers being successively formed loading period by loading period at which the organic wastes are loaded in the first loading opening through a daily routine, and the plurality of layers having different progress levels of decomposing carried out by a thermoduric bacteria flora under an aerobic condition in which a first air supply volume to the primary fermentation tank is controlled by the control device, the secondary fermentation tank includes a second ejecting device which ejects a second lowermost layer from a second ejecting opening provided in a bottom portion of the secondary fermentation tank as the secondary processed materials and which moves second stacked materials having the second lowermost layer eliminated therefrom downwardly in a horizontal condition after a process time of the second lowermost layer that is the lowermost layer in the second stacked materials has elapsed by a predetermined second process time in the secondary fermentation tank, the second stacked materials including a plurality of substantially horizontal layers stacked together, the plurality of layers being successively formed loading period by loading period through the second loading opening, and the plurality of layers having partially different progress levels of decomposing carried out by another thermoduric bacteria flora under an aerobic condition in which a second air supply volume to the secondary fermentation tank is controlled by the control device, and the deodorizing device includes: an acid pickling tank that performs acid pickling on evacuated gases from the primary fermentation tank and the secondary fermentation tank; a rinsing tank that performs rinsing on the gases having undergone acid pickling; and a microbial deodorizing tank that performs microorganism-deodorizing on the gases having undergone rinsing.

It is preferable that the first ejecting device should include a plurality of screw members each having a continuous spiral blades having a spiral pitch becoming narrow in a horizontal direction directed to the first ejecting opening, the plurality of screw members being arranged so as to be parallel to a bottom face of the primary fermentation tank and to have respective one ends protruding to an exterior of the primary fermentation tank from the first ejecting opening.

It is preferable that the second ejecting device should include a plurality of screw members each having a continuous spiral blades having a spiral pitch becoming narrow in a horizontal direction directed to the second ejecting opening, the plurality of screw members being arranged so as to be parallel to a bottom face of the secondary fermentation tank and to have respective one ends protruding to an exterior of the secondary fermentation tank from the second ejecting opening.

It is preferable that the useful product producing apparatus should further include a water supply device that sprinkles water to the second stacked materials depending on a water containing amount of the second stacked materials.

It is preferable that a temperature inside the fermentation device should be set to satisfy a condition in which a temperature produced by the thermoduric bacteria flora decomposing the organic wastes in the primary fermentation tank is higher than a temperature produced by the another thermoduric bacteria flora decomposing the primary processed materials in the secondary fermentation tank.

It is preferable that a moistening agent and/or the useful product should be loaded in the primary fermentation tank together with the organic wastes.

It is preferable that the organic wastes should be selected from the group consisting of: a fecal substance of animal; a waste produced through breaking-up of animal body; a waste produced when green things are cooked; a waste produced through a process of obtaining an effective ingredient from green things; and a waste produced through a process for environment preservation, and mixture thereof.

It is preferable that the waste produced through breaking-up of an animal body should include various tissues and bones of animal, and it is preferable that the waste produced through a process of obtaining an effective ingredient from green things should include various tissues of green things. It is preferable that the waste produced through a process of obtaining an effective ingredient from green things should be selected from the group consisting of; leaves, outer skins, and outer skin fibers of palm; shells and empty clusters of fruit; roots; and stocks.

It is preferable that the thermoduric bacteria floraehould comprise a plurality of bacteria which suitably grow in a temperature range from 50 to 90° C., and which partially decompose the organic wastes to produce primary processed materials. It is preferable that the another thermoduric bacteria floraehould comprise a plurality of bacteria which suitably grow in a temperature range from 40 to 70° C., and which decompose the primary processed materials to produce secondary processed materials.

It is preferable that the microbial deodorizing tank should include a plurality of layers each of which includes microorganism immobilized to peat moss, outer skin fibers of timber, outer shell fibers of seed of timber, empty clusters of seed, or a porous ceramic substrate.

It is preferable that the microorganism immobilized to the plurality of layers should be at least one kind of microorganisms selected from the group consisting of: 4pgm6 (Biotechnology center of National Institute of Technology and Evaluation deposit number: FERM P-17867); 4pg2 (Biotechnology center of National Institute of Technology and Evaluation deposit number: FERM P-17869); 4pg4 (Biotechnology center of National Institute of Technology and Evaluation deposit number; FERM P-17870); 4pgm2 (Biotechnology center of National Institute of Technology and Evaluation deposit number: FERM P-17865); 4pgm3 (Biotechnology center of National Institute of Technology and Evaluation deposit number FERM P-17866); 1pg2 (Biotechnology center of National Institute of Technology and Evaluation deposit number: FERM P-17868); and 4pgm8 (Biotechnology center of National Institute of Technology and Evaluation deposit number: FERM P-17871).

Moreover, it is preferable that the useful product should be selected from the group consisting of: fertilizer; feeding stuff; and fuel.

The present invention also provides a useful product producing method that includes: a primary process step of causing organic waste layers to be decomposed by a thermoduric bacteria flora under an aerobic condition in order to produce primary processed materials while moving the organic waste layers downwardly in a horizontal condition, the organic waste layers including substantially horizontal layers successively formed loading period by loading period at which organic wastes are regularly loaded in a first loading opening provided in an upper portion of a primary fermentation tank; a first take-out step of taking out a lowermost layer of the primary processed materials from a first ejecting opening provided in a bottom portion of the primary fermentation tank; a convey step of conveying the primary processed materials taken out from the primary fermentation tank to a second loading opening provided in an upper portion of the secondary fermentation tank; a secondary process step of causing the organic wastes not decomposed in the primary process step to be decomposed by another bacteria flora under an aerobic condition while moving secondary process layers downwardly in a horizontal condition, the secondary process layers including substantially horizontal layers stacked together and formed successively loading period by loading period at which the primary processed materials taken out in the first take-out step are loaded into the upper portion of the secondary fermentation tank through the convey step; and a second take-out step of taking out a lowermost layer of the secondary process layers from an ejecting opening provided in a bottom portion of the secondary fermentation tank.

It is preferable that the thermoduric bacteria floraehould comprise a plurality of bacteria which suitably grow in a temperature range from 40 to 90° C., and which partially decompose the organic wastes to produce primary processed materials. It is preferable that the another thermoduric bacteria floraehould comprise a plurality of bacteria which suitably grow in a temperature range from 40 to 70° C., and which decompose the primary processed materials to produce secondary processed materials.

It is preferable that in the secondary process step, water should be sprinkled to the secondary processed materials depending on a water containing amount of the secondary processed materials.

The present invention further provides a useful product that is produced through the above-explained useful product producing method.

It is preferable that the useful product should be selected from the group consisting of: fertilizer; feeding stuff; and fuel, and it is preferable that all of the thermoduric bacteria florae each should include a plurality of bacteria which can grow at a temperature from 40 to 90° C.

It is preferable that the organic wastes should be selected from one of the group consisting a fecal substance of animal; a waste produced through breaking-up of animal body; a waste produced when green things are cooked; a waste produced through a process of obtaining an effective ingredient from green things; a waste produced through a process for environment preservation; a leftover and a food disposed because of expiration of consumption date, and mixture thereof.

It is preferable that the fecal substance of animal should be selected from the group consisting of cow feces; horse feces; sheep feces; hog feces; deer feces; reindeer feces; elephant feces; camel feces; goat feces; buffalo feces; chicken feces; kangaroo feces; ostrich feces; rabbit feces; gator feces; and mixture thereof.

It is preferable that the waste produced through breaking-up of animal body should be selected from the group consisting of (1) various tissues and bones which are produced and disposed through breaking-up for a food processing; (2) various tissues which are produced and disposed through a cooking; and (3) a mixture thereof.

It is preferable that the waste produced through a process of obtaining an effective ingredient from green things should be selected from the group consisting of (1) various tissues which are produced and disposed through a food processing; (2) various tissues which are produced and disposed through a cooking; (3) residuals produced through an oil obtaining process; and (4) a mixture thereof.

It is preferable that the waste produced through a process of obtaining an effective ingredient from green things should be selected from the group consisting of leaves, branches, stocks, cortexes, roots, seeds of green things; outer skins of seeds; fruits; outer skins of fruits; flowers; flower buds; and stems; and mixture thereof, all of which are subjected to one treatment selected from the group consisting of vapor distillation; compression; and extraction.

It is preferable that the waste produced through a process of obtaining an effective ingredient from green things should be selected from the group consisting of leaves, outer skins, and outer skin fibers of palm; shells and empty clusters of fruit and seed; roots; and stocks; and mixture thereof. It is preferable that the waste produced through a process of environmental preservation should be selected from the group consisting of branches; forest thinning; underbrush; weeds of farmland cut for a purpose of pruning, and mixture thereof.

According to the configuration of the present invention, wastes loaded in a fermentation tank are decomposed by aerobic and thermophilic microorganisms, and are converted into useful products while remarkably reducing the volume. Moreover, the organic wastes to be processed are not limited to any particular ones.

Furthermore, according to the configuration of the present invention, the volume of air supplied to the fermentation tank can be remarkably reduced in comparison with conventional technologies, so that the amount of gases evacuated and the amount of heat released to the exterior of an apparatus by hot gases can be reduced.

Hence, the bad odor of the gases evacuated during the conversion process to the useful products can be deodorized by the above-explained deodorizing device, and the useful product producing apparatus can be configured to be small needing no large space and to be an energy-saving type.

Moreover, by using the producing apparatus of the present invention, various wastes can be efficiently processed, and fertilizers, feeding stuffs, and other useful products can be efficiently produced.

### BRIEF DESCRIPTION OF DRAWINGS

FIG. 1 is a diagram showing a general configuration of a useful product producing apparatus according to the present invention;

FIG. 2 is a diagram showing a general configuration of an air supply device installed in a fermentation tank of the apparatus shown in FIG. 1;

FIG. 3 is a conceptual diagram showing a configuration of a deodorizing device of the apparatus shown in FIG. 1;

FIG. 4 is a diagram showing a general configuration of the layout of a temperature sensor in the fermentation tank of the apparatus shown in FIG. 1;

FIG. 5 is a top view showing an ejecting device installed in the fermentation tank of the apparatus shown in FIG. 1;

FIG. 6 is a cross-sectional view showing a configuration of deodorizing tank in the deodorizing device shown in FIG. 3;

FIG. 7(A) to (C) are microscope photographs showing the forms of colonies formed by microorganisms (1pg2, 4pg2, 4pgm2, and 4pgm8) immobilized to the microbial deodorizing tank shown in FIGS. 1 and 3;

FIG. 8(A) to (C) are microscope photographs showing the forms of colonies formed by microorganisms (4pg4, 4pgm6, and 4pgm3) immobilized to a microbial deodorizing tank shown in FIGS. 1 and 3;

FIG. 9 is a diagram showing a conceptual configuration of a device for checking a temperature change in the fermentation tank;

FIG. 10 is a graph showing how temperature changes in the device shown in FIG. 4 depending on a location of a layer from primary fermentation to maturing (third fermentation);

FIG. 11 is a graph showing a change in a smell index in the deodorizing device of the apparatus shown in FIG. 4;

FIG. 12 is a graph showing a change in water contents of organic wastes in a primary fermentation tank of the apparatus shown in FIG. 1;

FIG. 13 is a graph showing a change in the weight of organic wastes before and after processing loaded in the apparatus shown in FIG. 1; and

FIG. 14 is a graph showing a result of thermal analysis of a fuel produced by the apparatus shown in FIG. 1.

### DETAILED DESCRIPTION

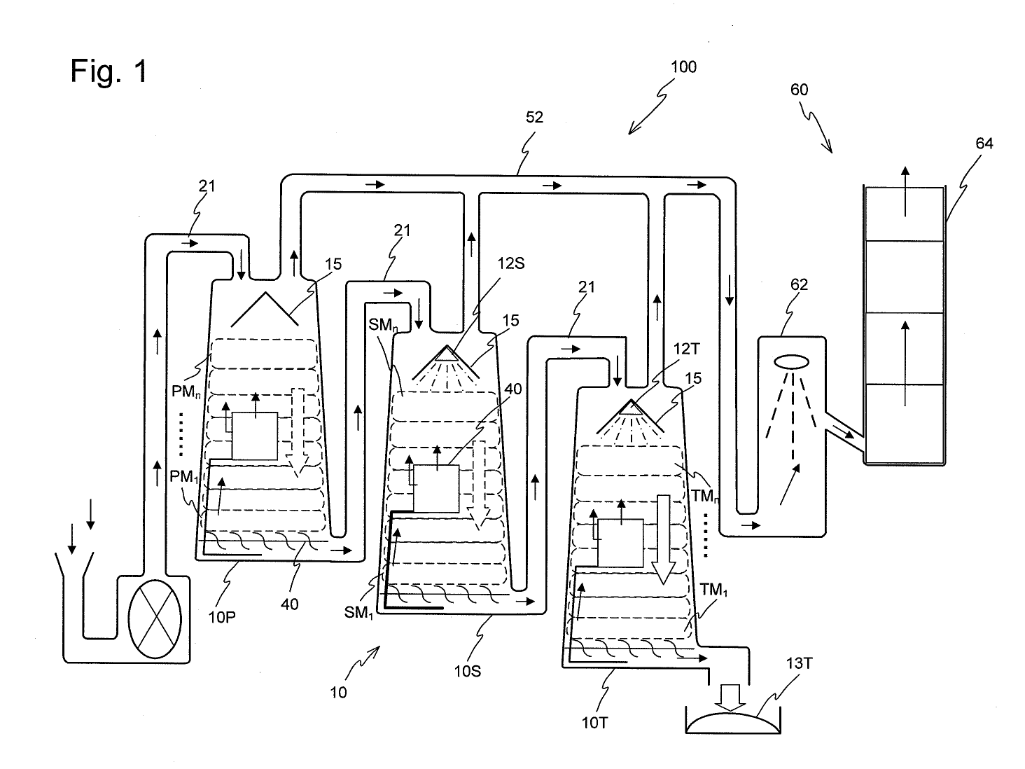

An explanation will be below given of an illustrative useful product producing apparatus of the present invention which includes three fermentation tanks (a primary fermentation tank 10P, a secondary fermentation tank 10S, and a third fermentation tank 10T) with reference to FIGS. 1 to 7. The same structural element will be denoted by the same reference numeral and the duplicated explanation will be omitted.

A useful product producing apparatus 100 (see FIG. 1) is a processing apparatus of decomposing organic wastes which includes (a) a fermentation device having plural processing tanks 10P, 10S, and 10T for decomposing organic wastes, (b) an air supply device 30 that forcibly supplies air to each of the plural processing tanks, (c) a deodorizing device 60 that deodorizes gases evacuated from each of the processing tanks, and (d) convey devices 21 which are provided between the primary fermentation tank and the secondary fermentation tank and between the secondary fermentation tank and the third fermentation tank, and which convey a primary processed material and a secondary processed material to the secondary fermentation tank and the third fermentation tank, respectively.

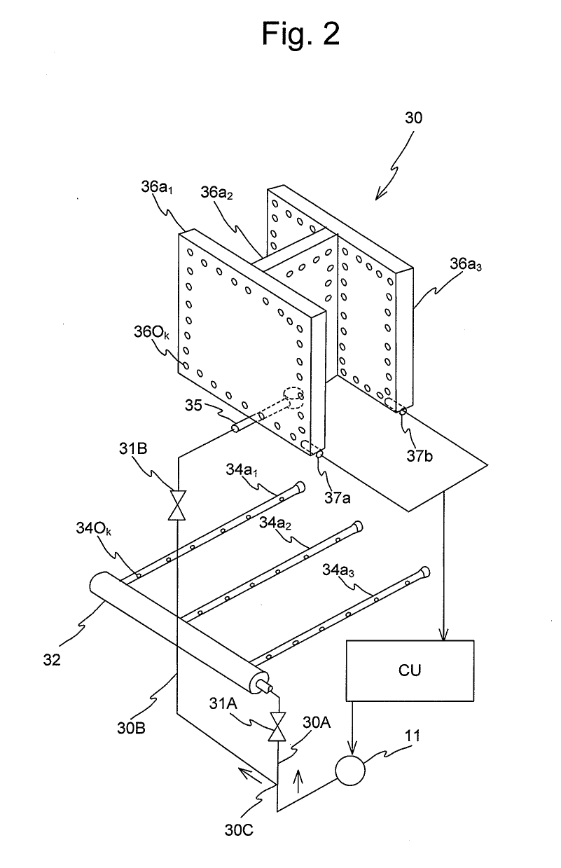

The air supply device 30 (see FIG. 2) includes (b1) a temperature sensor arranged inside the main body of each processing tank, (b2) plural spraying members 32 and 33 connected to an air pump 11 arranged outside the processing tanks, (b3) a timer and (b4) a temperature control device 37.

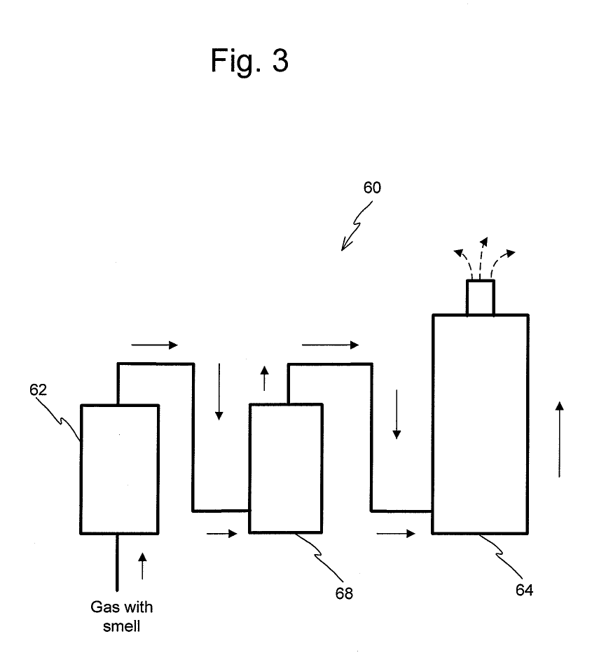

The deodorizing device 60 includes (c1) an acid pickling tank 62, and (c2) a microbial deodorizing tank 60. Gases produced when the organic wastes are fermented by heat-resisting bacteria florae in the fermentation tanks are collected from respective upper portions of the fermentation tanks 10P, 10S, and 10T, and go through the deodorizing device 60, and thus deodorized. Note that (as shown in FIG. 3) (c2) a rinsing tank 68 may be provided between (c1) the acid pickling tank 62 and (c2) the microbial deodorizing tank 60.

Moreover, (d) the convey devices 21 each comprise a flash conveyer, and are provided between the primary fermentation tank 10P and the secondary fermentation tank 10S, and between the secondary fermentation tank 10S and the third fermentation tank 10T, respectively. The primary processed material processed by the primary fermentation tank 10P is ejected from an ejection port, and is loaded in the secondary fermentation tank 10S through the flash conveyer.

As shown in FIG. 1, the side wall face of each fermentation tank 10P, 10S and 10T is not in a cylindrical shape with the same diameter, but is in a tapered shape so as to have an inclination from approximately 5 degrees to 20 degrees from the bottom of each fermentation tank to the top thereof. The upper portion of each fermentation tank is provided with (e) a loading opening for loading organic wastes therein, and the bottom portion of each fermentation tank is provided with (f) an ejecting opening for ejecting the processed material having undergone fermentation and an ejecting device 40 that is attached to the ejecting opening. Each fermentation tank is further provided with, below the loading opening, a loading duct that opens in direction and a spreading device 15 for forming a processed-material layer.

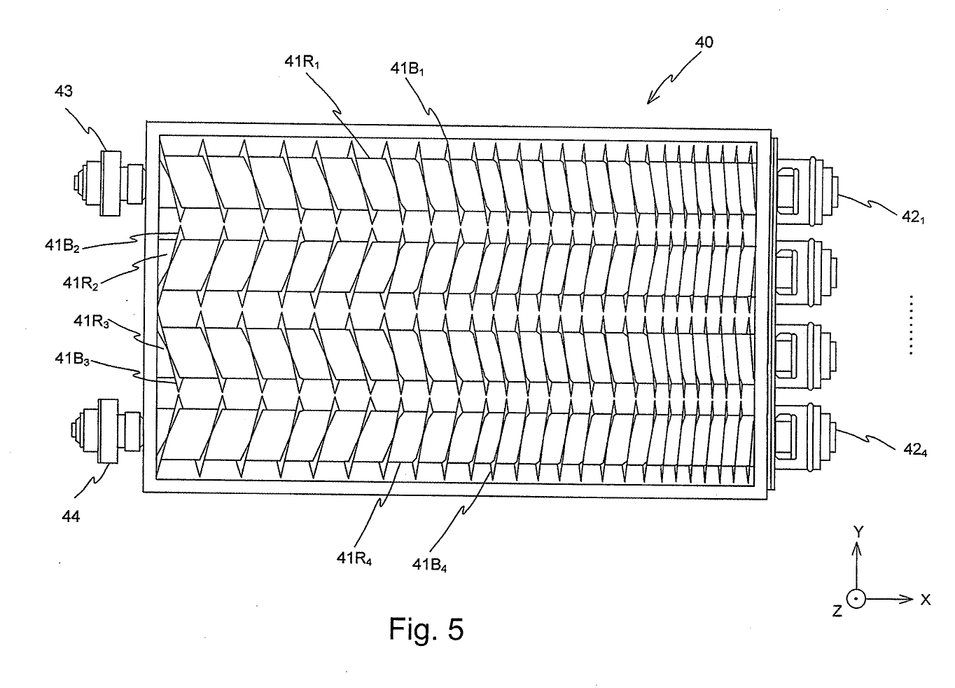

The above-explained (f) ejecting device 40 is used when the processed material having undergone each process completed is taken out from each fermentation tank layer by layer which is formed by such processed material. The ejecting device 40 includes four screw shafts 41R₁ to 41R₄ having continuous spiral blades 41B₁ to 41B₄, respectively, and two driving motors (unillustrated) that drive the screw shafts 41R₁ and 41R₄. The driving motors and the screw shafts 41R₁ and 41R₄ are connected to connecting members 43 and 44, respectively (see FIG. 5).

The continuous spiral blades 41B₁ to 41B₄ are formed on respective screw shafts 41R₁ to 41R₄ so as to have a pitch narrowed down gradually in the X direction. When the continuous spiral blades 41B₁ and 41B₄ are rotated by respective driving motors, the continuous spiral blades 41B₂ and 41B₃ facing with those spiral blades 41B₁ and 41B₄, respectively, are rotated so as to be meshed with respective spiral blades 41B₁ and 41B₄ together with the rotation thereof. Upon rotation of the pair of screw shafts, a processed-material layer PLₙ having undergone the process completed in the primary fermentation tank 10P and descended right above respective screw shafts 41R₁ to 41R₄ is fed in a horizontal condition to the X axis direction by the rotation of the screw shafts.

The primary processed material taken out from the primary fermentation tank 10P by the ejecting device 40 in this fashion is conveyed to the secondary fermentation tank 10S through the convey device 21, is loaded in the secondary fermentation tank 10S from the above thereof, and forms a secondary processed-material layer SLₙ.

Moreover, provided above the fermentation tanks 10S and 10T other than the primary fermentation tank 10P are (g) water supply devices 12S and 12T for supplying water to respective processed materials. A primary processed material PMₙ processed by the bacteria florae (hereinafter, referred to as “primary bacteria florae” in some cases) formed as will be discussed later in the primary fermentation tank 10P, conveyed to the secondary fermentation tank 10S by the convey device and loaded from the loading opening is provided with water supply from the water supply device 12S, and is to be processed by bacteria florae (hereinafter, referred to as “secondary bacteria florae” in some cases) formed in the secondary fermentation tank 10S as will be discussed later.

A secondary processed material SMₙ processed in the secondary fermentation tank 10S is conveyed to the third fermentation tank 10T through the same fashion as explained above, is processed by bacteria florae (hereinafter, referred to as “third bacteria florae” in some cases) formed in the third fermentation tank 10T, and is ejected as a useful product TMₙ.

The reason why the primary fermentation tank 10P is not provided with a water supply device is that the water content of the organic wastes loaded in the primary fermentation tank lop is substantially 80%, which is sufficient for the growth of microorganisms. In contrast, respective water content of the processed materials in the fermentation tanks 10S and 10T other than the primary fermentation tank decrease as the fermentation by microorganisms progresses. When the water content of each processed material decreases to substantially 35% to 40%, the growth of microorganism stops and the fermentation is terminated. Because of such a reason, the water supply devices 12S and 12T are provided above the fermentation tanks other than the primary fermentation tank for supplying water.

By employing such device configuration, a fermentation raw material (organic waste) RM put in each fermentation tank in a layer form as will be discussed later contacts air supplied from the bottom of the primary fermentation tank 10P, and thus the microorganisms grows.

Moreover, respective ceilings of the fermentation tanks 10P, 10S, and 10T are provided with (h) degassing openings for feeding gases produced through the fermentation process to the deodorizing device, and such gases are fed to the deodorizing device 60 to be discussed later through a degassing pipe 52 connected to each of the degassing openings.

As explained above, the deodorizing device 60 includes (c1) the acid pickling tank 62 and (c3) the microbial deodorizing tank 64. The gases are first blown in from the bottom of the acid pickling tank 62, and contact acids flowing down through the wall surface from the upper part of the acid pickling tank 62. The alkaline components are neutralized by this contact. The rinsing tank 68 (see FIG. 3) may be provided as needed which allows the gas passing through the acid pickling tank 62 to flow through.

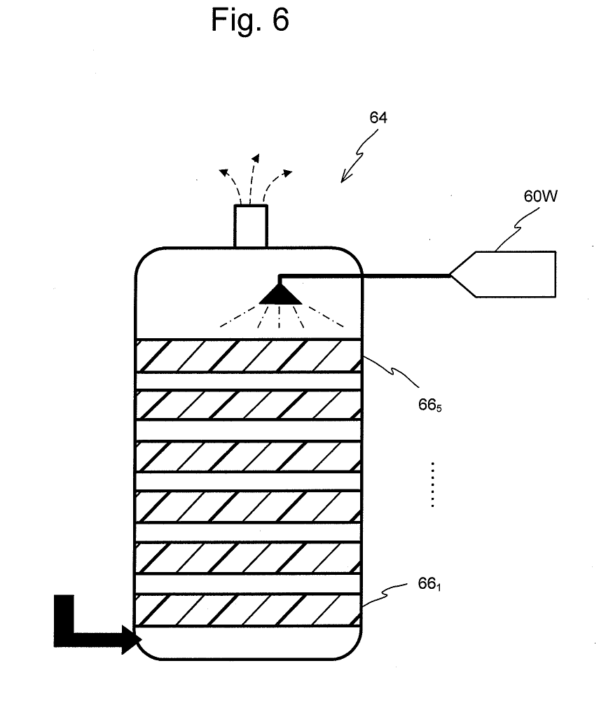

Next, the gases passing through the acid pickling tank 62 are fed to the microbial deodorizing tank 64. The microbial deodorizing tank 64 uses plural kinds of bacteria having a deodorizing function (see FIG. 6). Such bacteria are immobilized as explained above, thereby forming plural microorganism deodorizing layers 66₁ to 66₅ in the microbial deodorizing tank 64.

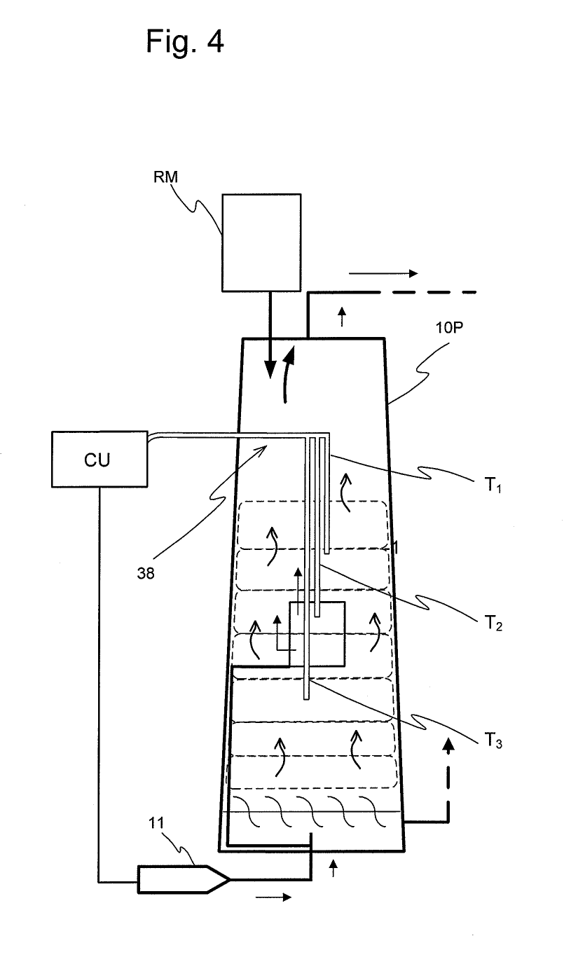

The bad odor, smell, components contained in the gases passing through the acid pickling tank 62 are decomposed by the microorganisms immobilized in the microbial deodorizing tank 64, and are released to the atmosphere (see FIG. 4).

The air supply device 30 that supplies air to the fermentation tank according to the present embodiment has two air pipe systems 30A and 30B, the air pump 11 that supplies air to those pipe systems, and a control unit CU that controls the air pump 11 based on the internal temperature of the fermentation tank detected by temperature sensors 37a and 37b.

Pumping of air from the air pump 11 is not limited to any particular scheme, but is, for example, a blower scheme, or a piston scheme.

Air pipes connected to the air pump 11 are divided into the pipe system 30A connected to the spraying member 34 and the pipe system 30B connected to a spraying system 36 at a branching point 30C. Airflow adjusting valves 31A and 31B are provided at respective predetermined locations in the halfway of respective pipe systems 30A and 30B.

The spraying member 34 connected to an end of the pipe system 30A includes plural pipe members 34a₁ to 34aⱼ (where j=1, 2, and 3) arranged between the bottom of the fermentation tank and the screw shafts so as to be parallel with such screw shafts. The pipe members 34a₁ to 34aⱼ each have spraying orifices 34O₁ to 34Oₖ (where k=1 to n) arranged and distributed in the +Z axis direction, and respective one ends of such pipe members are connected to a common air chamber 32.

In the pipe system 30A, air fed from the air pump 11 passes through the airflow adjusting valve 31A and the air chamber 32, reaches the spraying member 34, and is supplied to the portion closest to the bottom of each fermentation tank.

Conversely, the spraying member 36 connected to the pipe system 30B includes three hollow panels 36a₁ to 36a₃ connected in an H shape. The X-Y cross section of the spraying member 36 in the −Z direction is arranged at a location in the +Z direction of the ejecting device 40 and at substantially middle of each fermentation tank (see FIG. 1). As shown in FIG. 2, the spraying member 36 is fixed to at least four locations of the inner wall so as to partition the internal space of each fermentation tank in the horizontal direction.

The spraying member 36 has a joint port 35 provided at an end in the −Z direction, and the pipe system 30B is connected thereto.

Moreover, respective hollow panels 36a₁ to 36a₃ configuring the spraying member 36 are formed with multiple spray orifices 36O₁ to 36Oₖ. Air supplied from the air pump 11 passes through the pipe system 30B provided with the airflow adjusting valve 32B, reaches the spraying member 36, and is blown out from the spraying orifices 36O₁ to 36Oₖ.

The amount of air supplied to each fermentation tank is the sum of the amount of air supplied to the lowermost portion of each fermentation tank by the spraying member 34 and the amount of air supplied from the middle portion of each fermentation tank by the spraying member 36, and the ratio of respective supply amounts is adjusted by the airflow adjusting valves 31A and 31B.

The control unit CU further has a timer function and a temperature setting function, and as shown in FIG. 4, is further connected to a temperature sensor 38. In the present embodiment, three thermometers T₁ to T₃ are used as the temperature sensor 38.

As shown in FIG. 4, the temperature sensor 38 is set at a predetermined location directed from the upper portion of the fermentation tank toward the −Z direction. For example, thermometers T₁ (38₁), T₂ (38₂), and T₃ (38₃) are set at respective locations 2/3, 1/2, and 3/1 from the upper portion of the fermentation tank in order to measure the temperatures at respective layers, and measured temperature information is transmitted to the control unit CU. The control unit CU increases/decreases the pressure at the time of pumping air from the air pump 11 based on the temperature information, thereby adjusting the temperature inside the fermentation tank 10P. More specifically, when fermentation has the temperature rising faster than expected pace, the pumping pressure is increased. Conversely, when the fermentation is slower than expected pace, the pumping pressure is decreased. The same control is performed on the other fermentation tanks 10S and 10T.

The temperature sensor 38 may be configured by a thermocouple sensor instead of the thermometer as explained above, and plural thermocouple sensors may be connected in parallel with each other. According to such a configuration, even if one of the thermocouple sensors configuring the temperature sensor breaks down, it is possible to keep detecting the temperature inside the fermentation tank through the other thermocouple sensors connected in parallel.

Air supplied into each fermentation tank as explained above passes through the layer of organic wastes, goes thereabove, and further gently goes up along the inner wall of the fermentation tank tapered at an angle of substantially 5 to 20 degrees. Because the air gently flows inside the fermentation tank, the environment that allows the aerobic microorganisms to grow is formed.

The growth of multiple microorganisms begins in the fermentation tank which maintains appropriate humidity and temperature thereinside. The grown microorganisms form bacteria florae, and when decomposing and fermentation of the fermentation raw materials (organic wastes) begin, heat is generated so that the temperature inside each fermentation tank starts rising (see FIG. 1).

Air supplied from the spraying members 34 and 36 and blown up from the bottom contacts the heat generated at the time of fermentation by microorganisms, and heated air slowly moves to the upper portion of the primary fermentation tank 10P, so that a heat accumulation portion is formed at the upper portion of the primary fermentation tank 10P by the hot air. Hence, in the primary fermentation tank 10P, a temperature distribution is formed so that it gradually becomes a lower temperature from +Z direction to the −Z direction. Accordingly, microorganisms appropriately living in each temperature range grow up in such a temperature distribution.

When the temperature rises in respective fermentation tanks are compared, the temperature rise in the primary fermentation tank is the largest, and the temperature of the heat accumulation portion at the uppermost portion of the primary fermentation tank reaches substantially 80 to 90° C. By repeating fermentation in the secondary fermentation tank and then in the third fermentation tank, the temperature rise gradually becomes small, and respective temperatures of the heat accumulation portions become around 70° C. and around 50° C. Hence, microorganisms grown in respective fermentation tanks become different kinds naturally, and the bacteria florae formed by respective grown microorganisms also become different kinds.

Next, an explanation will be given of decomposing and fermentation of the organic wastes in each fermentation tank.

First, the organic wastes loaded from the loading opening provided in the upper portion of the primary fermentation tank 10P on the first day form an organic waste layer RL₁. The organic wastes loaded on the second day form a next organic waste layer RL₂, and by repeating this procedure, an organic waste layer RL is formed in the primary fermentation tank 10P day by day when the organic wastes are loaded therein.

An example of the organic wastes can be selected from a group of the followings or the mixture thereof: a fecal substance of an animal; a waste produced through breaking-up of an animal body; a waste produced when green things are cooked; a waste produced through a process of obtaining effective ingredients from green things; the waste produced through a process for environment preservation; and a leftover and a food disposed because of expiration of consumption date. However, the organic wastes are not limited to the above examples.

More specifically, examples of the fecal substance of an animal are feces of cow, horse, sheep, deer, reindeer, camel, kangaroo, and gator produced at a stock farm, etc., feces of pig produced at a hog farm, feces of chicken produced at a chicken farm, feces of elephant, feces of goat, feces of buffalo, feces of rabbit, and feces of other animals and the mixture thereof.

Moreover, the wastes produced through breaking-up of an animal body like a food processing can also be used as the organic wastes. An example of such wastes can be selected from a group of the followings: (1) skins, outer shells, tendon, visceral tissues, other various tissues and bones including gristle of animals like mammal, reptilian, fish, and shellfish which are produced and disposed through breaking-up for a food processing; (2) outer skins, fat tissues, and various tissues not appropriate to eat like muscular substances contained in chopped flesh and meat, and (3) the mixture thereof.

The wastes produced through a process of obtaining effective ingredients from green things include (1) various tissues produced and disposed through a food processing, such as leaves of carrot, and outer skins of corn; and (2) various tissues produced and disposed through cooking, such as leaves of radish, skins of carrot, potato, radish, etc., the internal seeds of pumpkin and cucurbitaceous vegetable, shells of green soybean and broad bean, skins of fruits, such as watermelon, melon, orange, and grape, and cores thereof including seeds, and cores of corn without grains, and hull of eggplant.

Furthermore, examples of the wastes produced through a process of obtaining effective ingredients from green things are leaves and branches of green things like blue gum, stocks of cypress and cedar, tree bark and roots thereof, seeds and the outer skins thereof like ambrette seed, fruits and the outer skins of fruits, such as orange, lemon, grapefruit, etc., flowers, buds, and stems of rose, lavender, etc., having undergone steam distillation, compression and extraction in order to obtain, for example, essential oils. The examples also include leaves, outer skins, outer-skin strings of palm tree, shells of fruits and seeds, empty clusters, roots, and stocks of fruit and seeds, and oil residuals derived from seeds of Jatropha.

Examples of wastes produced through the process for environment preservation are branches of street trees and garden trees cut for the purpose of pruning, forest thinning and underbrush produced at mountain forests, and weeds grown at farmlands, grass turfs, and under the street trees.

Furthermore, branches cut out for pruning, woodchips and sawdust produced when the forest thinning is cut to a predetermined length, and tree barks, woodchips, sawdust, etc., produced at the time of lumber sawing can be used as a moistening material which adjusts the water contents of the organic wastes in the primary fermentation tank. When such materials are used as the moistening material, it is preferable if the moistening material should be dried because the absorption amount of water is large. When the moistening material is put in the primary fermentation tank beforehand, adjustment of water amount is facilitated.

The primary fermentation tank 10P is provided with the air supply device 30 that forcibly supplies air as explained above, and air is supplied after the organic waste layer RL₁ is formed. Hence, the interior of the primary fermentation tank 10P becomes an aerobic condition, and plural microorganisms start growing substantially simultaneously.

Decomposing and fermentation of the organic wastes progress together with the growth of the microorganisms, and heat is generated. This heat contacts air blown out from the bottom of the primary fermentation tank 10P, heats up the air, and thus the heat accumulation portion at a temperature of substantially 80 to 90° C. is formed in the upper portion of the primary fermentation tank 10P.

Accordingly, only bacteria which can grow up under a high temperature condition from substantially 80 to 90° C. at the upper portion of the primary fermentation tank, and such bacteria form bacteria florae. In the primary fermentation tank, the temperature decreases from the top to the bottom, and a temperature distribution like substantially 80 to 90° C. at maximum and substantially 40° C. at minimum is formed.

The organic waste layers RLₙ (where n=1 to 10) are formed successively in the order of loading-in of the organic wastes, and the organic wastes forming such layers are decomposed by the bacteria florae, so that the layers move down to the bottom of the primary fermentation tank 10P (in the bottomwise and −Z direction) while decreasing respective volumes and water contents and being maintained in substantially horizontal without being collapsed.

In the primary fermentation tank 10P, only resources which can be decomposed by the bacteria florae in the organic waste layer RLₙ are decomposed, and a primary-processed-material layer is formed. When a process is carried out for 10 days in the primary fermentation tank 10P, the decomposing percentage of the organic wastes loaded on the first day is substantially 50%.

The primary-processed-material layers PLₙ are taken out by the ejecting device 40 from the ejecting opening in the order of the formation of respective layers, and are conveyed to the secondary fermentation tank 10S by the convey device 21.

The ejecting device 40 (see FIG. 5) includes, as explained above, the four screw shafts 41R₁ to 41R₄ each having the continuous spiral blade, and the two driving motors for driving such screw shafts.

As explained above, the ejecting opening is provided in a predetermined location of the side wall contacting the bottom of each fermentation tank, and each opening is provided with the duct. The primary fermentation tank 10P and the secondary fermentation tank 10S use respective ducts in order to put respective processed materials into respective containers that convey such processed materials to the secondary fermentation tank 10S and the third fermentation tank 10T, respectively. The third fermentation tank 10T uses such a duct in order to take out the useful products (hereinafter, referred to as “composts” in some cases) produced thereinside. The taken-out composts are moved to a reserving container or loaded in the primary fermentation tank 10P as return composts together with the organic wastes.

The ejecting device 40 has the screw shafts 41R₁ to 41R₄ so as to be in parallel with the bottom of the primary fermentation tank 10P and to have respective one ends protruding outwardly of the primary fermentation tank 10P through the ejecting opening. Respective ends of the screw shafts 41R₁ to 41R₄ are provided with gears, and the two screw shafts among the four screw shafts 41R₁ to 41R₄ form a pair, and the pair of adjoining screw shafts mesh with each other through respective gears. Hence, when the driving motor rotates either one of the screw shafts 41R₁ and 41R₄ in a certain direction, the screw shafts 41R₂ and 41R₃ forming the pair with respective screw shafts 41R₁ and 41R₄ rotate in a reverse direction to the driven screw shaft, and objects are tucked between the pair of screw shafts.

Moreover, as shown in FIG. 5, the continuous spiral blades 41B₁ to 41B₄ of respective four screw shafts 41R₁ to 41R₄ have spirals with a pitch becoming narrow along the X axis direction. This enables the ejecting device to take out the whole primary-processed-material layer PLₙ from the ejecting opening with the processed-material layer being horizontal, and to prevent the primary processed materials from being left at a side apart from the ejecting opening.

The taken-out layer PL of the primary processed materials is conveyed to the secondary fermentation tank 10S by the above-explained convey device layer by layer.

Next, the primary processed materials PM loaded in the secondary fermentation tank 10S form the primary-processed-material layer PL₁ like the above-explained organic waste layer RL₁, and are converted into secondary processed materials SM in the secondary fermentation tank 10S through the same procedure as that of the fermentation in the primary fermentation tank 10P.

That is, as explained above, after water is supplied, microorganisms begin to grow through the same mechanism as explained above, and a heat accumulation portion of substantially around 70° C. is formed at the upper portion of the secondary fermentation tank 10S. Thereafter, a temperature distribution is formed which decreases the temperature toward the bottom of the secondary fermentation tank 10S. Because the lower portion of the secondary fermentation tank 10S becomes substantially 40° C., the temperature distribution of the secondary fermentation tank 10S has the maximum temperature which is substantially 70° C. and the minimum temperature which is substantially 40° C.

The primary processed materials PLₙ (where n=1 to 10) form layers in the order of loading-in to the secondary fermentation tank 10S, decomposed by the above-explained bacteria florae, and move down to the bottom of the secondary fermentation tank 10S while gradually reducing its volume and water contents.

In the secondary fermentation tank 10S, also, only resources which can be decomposed by the bacteria florae among the primary-processed-material layers PLₙ are decomposed, and the secondary processed materials are formed. The secondary processed materials are taken out from the ejecting opening by the ejecting device 40 in the order of loading-in, and are conveyed to the third fermentation tank 10S by the convey device 21.

Hence, the bacteria florae are formed by microorganisms appropriate for such a temperature distribution, and the primary-processed-material layers PLₙ are stacked in the order of loading-in to the secondary fermentation tank 10S, are subjected to fermentation and decomposing by the microorganisms, and move in the downward direction of the secondary fermentation tank 10S while maintaining a horizontal condition. When a process is carried out for 10 days in the secondary fermentation tank 10S, substantially 50% of the unprocessed materials contained in the primary processed materials are decomposed, and become secondary processed materials.

The secondary processed materials SM taken out from the ejecting opening are conveyed to the third fermentation tank 10T like the primary processed materials PM, loaded in the third fermentation tank 10T through the loading opening, and form processed-material layers TLₙ. Thereafter, such processed-material layers are supplied with water from the water supply device 12T, processed by the third bacteria florae formed by grown microorganisms, subjected to fermentation and decomposing by the microorganisms through the same procedure as explained above for the secondary fermentation tank 10S, and move down to the bottom of the third fermentation tank 10T.

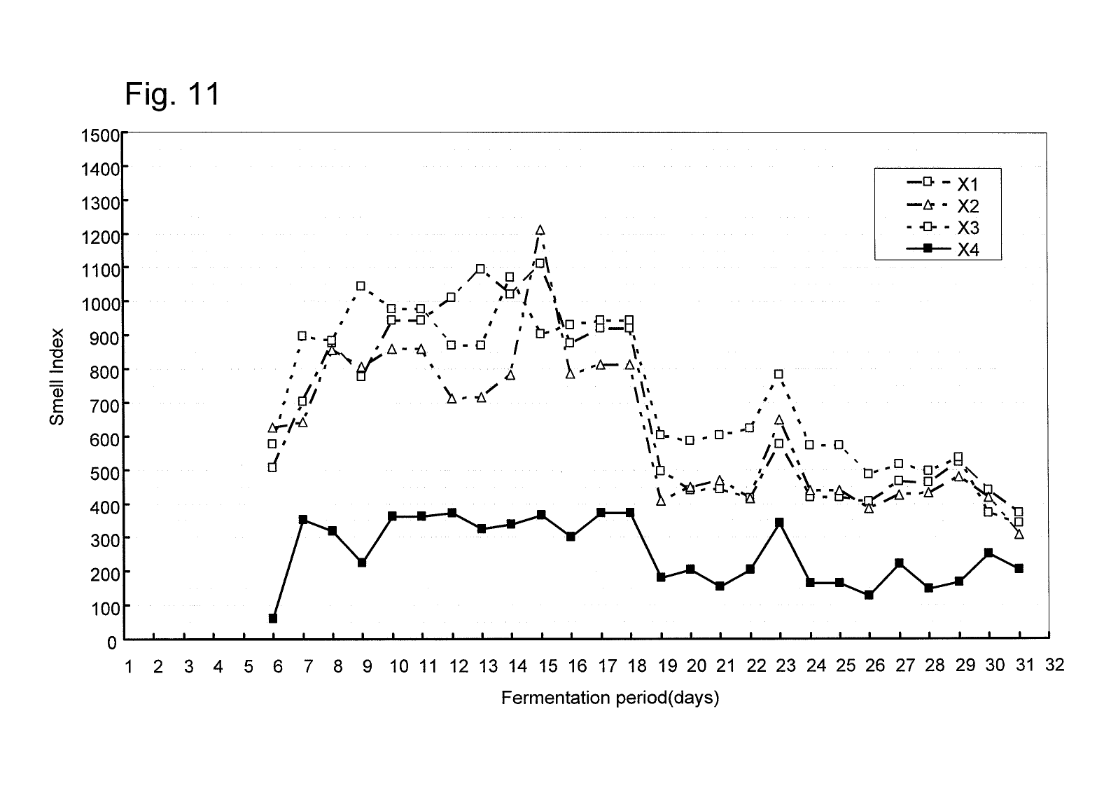

The water contents of the organic wastes which was substantially 65 wt % when the organic wastes were loaded in the primary fermentation tank 10P decreases to substantially 40% (see FIG. 11 to be discussed later) through the processes by the bacteria florae in the primary fermentation tank 10P and in the secondary fermentation tank 10S. When the water content becomes substantially 35 to 40 wt %, most microorganisms become a dormant state. Accordingly, water is supplied from the water supply device 12T in the third fermentation tank 10T after the secondary processed materials SM are loaded therein.

The heat accumulation portion of substantially 50° C. is formed at the upper portion of the third fermentation tank 10T, and the temperature of the lower portion thereof becomes substantially 40° C. Accordingly, in the third fermentation tank 10T, microorganisms appropriate for such a temperature distribution grow, and the third bacteria florae are formed by such microorganisms.

When the process in the third fermentation tank 10T is carried out for 10 days, there is a difference depending on the compositions of the organic wastes, but substantially 20 to 70% of the unprocessed materials contained in the secondary processed materials SM are decomposed. The third processed materials TM processed in the third fermentation tank 10T are taken out by the ejecting device 40, and are stored as ripened composts.

When the processes are carried out for substantially 30 days in the primary fermentation tank 10P to the third fermentation tank 10T as explained above, the third processed materials TM containing substantially 10 to 30% of undecomposed materials of the organic wastes originally loaded are obtained.

Some of the obtained third processed materials TM can be loaded in the primary fermentation tank 10P together with the organic wastes RM as explained above.

In the primary fermentation tank 10P, as explained above, the organic wastes are decomposed and fermented by the bacteria florae and the primary processed materials PM are formed, but gases with smell are often produced through this process. Such gases contain ammonium and sulfur-containing compounds, and it is not an appropriate process for letting such gases directly released to the atmosphere. Hence, such gases are collected from the degassing opening provided in the ceiling of the primary fermentation tank 10P, go through the degassing pipe joined with the degassing opening, and are fed to the deodorizing device 60 (see FIG. 1).

The deodorizing device 60 includes the acid pickling tank 62 and the microbial deodorizing tank 64. First, the gases are blown into the bottom of the acid pickling tank 62, and contact the acid flowing down from the upper portion of such a tank. Such contact causes the alkaline components to be neutralized (see FIG. 1). In the case of FIG. 1, the acid is supplied as mists from the upper portion of the acid pickling tank, but may flow through the inner wall thereof.

The gases passing through the acid pickling tank 62 are successively fed to the microbial deodorizing tank 64 and deodorized. The microbial deodorizing tank 64 (see FIG. 6) includes plural immobilized layers 66₁ to 66₅ to be discussed later where plural kinds of microorganisms are immobilized, and a water supply device 60W that supplies water to the microorganisms.

The gases passing through the acid pickling tank 62 are blown into the bottom of the microbial deodorizing tank 64, and pass through the immobilized layers 66₁ to 66₅. At this time, the immobilized bad odor components, such as ammonium, thiol, and skatole, are decomposed by the microorganisms to be discussed later. Thereafter, the deodorized gases are released to the atmosphere through an evacuating opening provided in the upper portion of the microbial deodorizing tank 64.

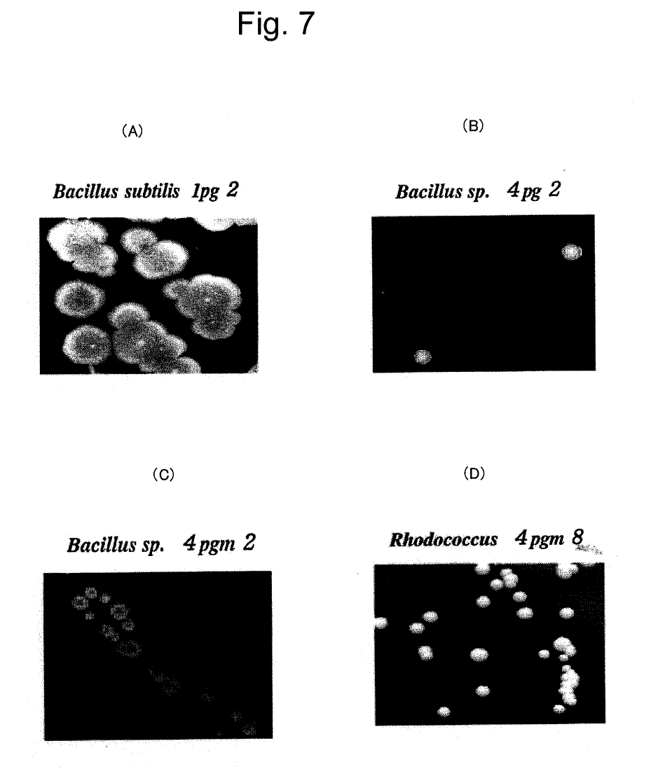

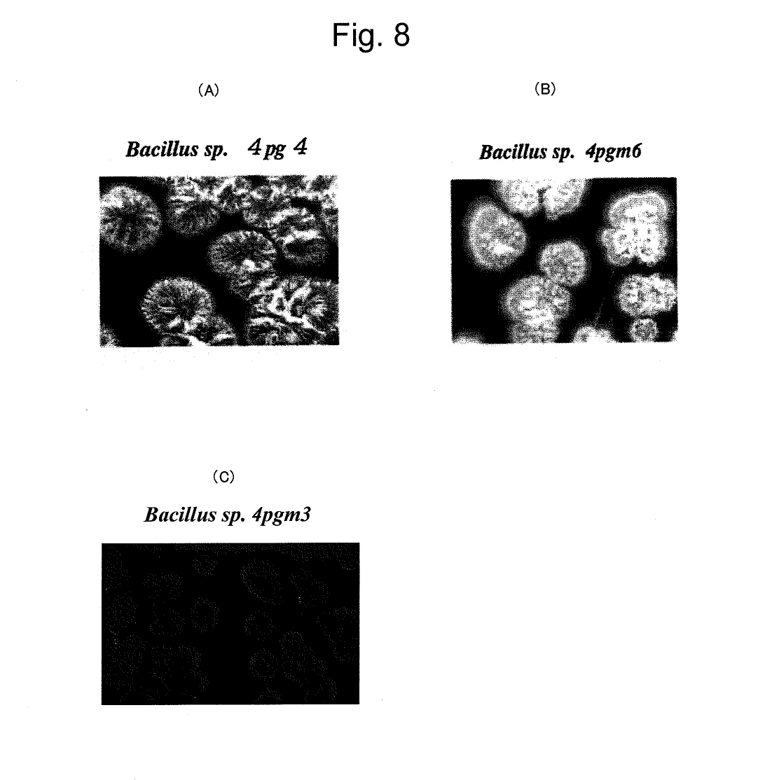

Examples of the microorganisms used for the microbial deodorizing tank 62 are 4pgm6 (Biotechnology center of National Institute of Technology and Evaluation, deposit number: FERM P-17867), 4pg2 (Biotechnology center of National Institute of Technology and Evaluation, deposit number: FERM P-17869), 4pg4 (Biotechnology center of National Institute of Technology and Evaluation, deposit number: FERM P-17870), 4pgm2 (Biotechnology center of National Institute of Technology and Evaluation, deposit number: FERM P47865), 4pgm3 (Biotechnology center of National Institute of Technology and Evaluation, deposit number FERM P-17866), 1pg2 (Biotechnology center of National Institute of Technology and Evaluation, deposit number: FERM P47868), and 4pgm8 (Biotechnology center of National Institute of Technology and Evaluation, deposit number: FERM P-17871). Respective bacteriological characteristics of such microorganisms are shown in tables 1 and 2 below. Moreover, the forms of colony formed by each bacteria are shown in FIGS. 7A to 7D and FIGS. 8A to 8C.

When seven kinds of such microorganisms are used, the deodorizing effect of gases containing bad odor components produced through fermentation and decomposing of the organic wastes becomes high, and in particular, the elimination effect of ammonium odor is high.

TABLE 1

| Bacteria No. | 1pg2 | 4pg2 | 4pgm2 | 4pgm8 |
|---|---|---|---|---|
| Deposit No. | FERM P-17868 | FERM P-17869 | FERM P-17865 | FERM P-17871 |
| CultivationTemp. (° C.) | 30 | 30 | 30 | 30 |
| Cell shape | Rod (0.8 × 2-3 μm) | Rod (0.8 to 1 × 3 to 5 μm) | Rod (0.8 × 5 to 10 μm) | Rod (0.6 × 1 to 1.2 μm) |
| Gram stain | Unstable | Unstable | − | + |
| Spore formation | + | + | + | − |
| Motility | + | + | − | − |
| Colony formation | Irregular | Irregular | Circular | Circular |
| | Periphery | Periphery ripple | Periphery ripple | Whole |
| | filamentous | Low convex | Low convex | periphery smooth |
| | Convex | Slightly grazed | Slightly grazed | Convex |
| | Matte | Pale yellow | Cream color | Grazed |
| | Cream color | | | Orange |
| Growth Temp. (° C.) | NT | NT | NT | NT |
| Catalase | + | + | + | + |
| Oxydase | − | + | + | − |
| O/F test | − | − | − | − |
| Tyrosine decomposition | − | − | − | |
| Casein decomposition | + | − | − | |
| Starch decomposition | − | − | − | |
| Hippuric acid decomposition | − | − | − | |
| Anaerobic growth | − | − | − | |
| Growth at 55° C. | − | NT | NT | |
| Identification result | Bacillus | Bacillus | Bacillus | Coryneform |
| | | | | bacterium |

TABLE 2

| Bacteria No. | 4pg4 | 4pgm3 | 4pgm6 |
|---|---|---|---|
| Deposit No. | FERM P-17870 | FERM P-17866 | FERM P-17867 |
| CultivationTemp. (° C.) | 30 | 30 | 30 |
| Cell shape | Rod (0.8 to 1 × 2 to 3 μm) | Rod (1 to 1.2 × 2 to 3 μm) | Rod (1 to 1.5 × 2 μm) |
| Gram stain | + | + | Indefinite |
| Spore formation | + | + | + |
| Motility | − | − | + |
| Colony formation | Irregular | Irregular | Irregular |
| | Periphery ripple | Periphery ripple | Periphery ripple |
| | Surface wrinkle | Surface wrinkle | Raised wrinkle |
| | Matte | Matte | Matte |
| | Cream color | Cream color | Cream color |
| Growth Temp. (° C.) | NT | NT | NT |
| Catalase | + | + | + |
| Oxydase | + | −(+w) | − |
| O/F test | + | + | + |
| Tyrosine decomposition | − | − | − |
| Casein decomposition | + | + | + |
| Starch decomposition | − | − | − |
| Hippuric acid | − | − | − |
| decomposition | | | |
| Anaerobic growth | − | − | − |
| Growth at 55° C. | NT | NT | NT |
| Identification result | Bacillus | Bacillus | Bacillus |

These bacteria can be obtained from Bio GIKEN INDUSTRIES, Co., Ltd., (product name: bacterial secondary stock).

The culture medium used for the growth of such bacteria can be obtained by, for example, dissolving predetermined amounts of commercially available Malt Extract, Yeast Extract and glucose into distilled water, and by adjusting the pH thereof to substantially 7.3 using alkaline like sodium hydroxide.

The culture medium prepared as explained above is separated evenly and poured into culture bottles with a predetermined volume, subjected to sterilization by using an autoclave, and stocked in room temperature. A bacterial secondary stock is inoculated to a culture medium in the culture bottle within a clean bench, and is cultured for a predetermined period using a culture device like a rotary shaker, thereby obtaining a suspension of microorganisms.

Next, predetermined amounts of KH₂PO₄, CaCl₂.2H₂O, MgSO₄.7H₂O, CoCl₂.6H₂O are dissolved in a predetermined amount of water in order to prepare a mineral liquid, and such a mineral liquid is stocked in a room temperature.

An explanation will be below given of an example case in which peat moss is used as the immobilizer for the microbial deodorizing tank.

A pack of commercially available peat moss (substantially 50 L, made in Canada or Europe and rich in fiber) is taken out from the pack, flexed, spread, and sprayed with 1 L of the above-explained mineral liquid per a pack of peat moss. Next, the above-explained culture liquid is turned upside down and mixed, and 200 to 300 mL of such liquid per a pack of peat moss is sprayed.

The immobilized layer prepared as explained above is filled in the container for the microbial deodorizing tank, and is used as the microbial deodorizing tank.

Examples of the immobilizer are outer skin fibers of timber, outer shell fibers of seed of timber, empty clusters of seed, porous ceramics, diatomaceous earth, active charcoal, zeolite particles, and sterilized soil.

Examples of the outer skin fibers of timber and the outer shell fibers of seed of timber are ones derived from timbers used for obtaining oils like palm tree. Moreover, examples of the empty clusters of seed are palm and Jatropha after oils are extracted. Such outer skin fibers, outer shell fibers and empty clusters are torn into pieces as needed.

When the immobilizer for immobilizing the bacteria is peat moss, active charcoal, powder zeolite, sterilized soil, or pieces of empty cluster, a punching metal, a net, etc., are sandwiched therebetween as needed in order to form an immobilizing layer, and can be filled in a column.

The above-explained bacteria may be cultured kind by kind, and may be separately immobilized to the immobilizer, or plural kinds of such bacteria may be immobilized together. Moreover, the above-explained immobilizers may be formed as individual layers with a desired thickness, soaked in the culture liquid of any one of the bacteria cultured separately, and may be stacked together in a desired order to form the microorganism deodorizing layer. It is preferable that all seven kinds of bacteria should be used in the deodorizing tank from the standpoint of good deodorizing effect. The number of layers is not limited to any particular number, but it is preferable if it is five or so from the standpoint of maintenance and management.

The microorganisms in the microbial deodorizing tank 64 are maintained so that the water contents of the immobilizer become 60% to 80% by supplying water. However, when the volume of the immobilizer decreases and the deodorizing effect becomes poor because of deterioration of the immobilizer, the deodorizing layer may be replaced with a new layer produced as explained above. In general, it is preferable that such a layer should be replaced with another one for each three months or so from the standpoint of deodorizing effect.

By allowing the gases to pass through the deodorizing device employing the above-explained configuration, such gases with smell produced when the organic wastes, the primary processed materials, and the secondary processed materials are processed in respective fermentation tanks can be deodorized, which enables the gases passing through the deodorizing device to be released to the atmosphere.

### EXAMPLES

Examples of the present invention will be explained below in order to explain the present invention in more detail, but the present invention is not limited to the following examples.

### First Example

### Change in Temperature and Odor in Fermentation Tank

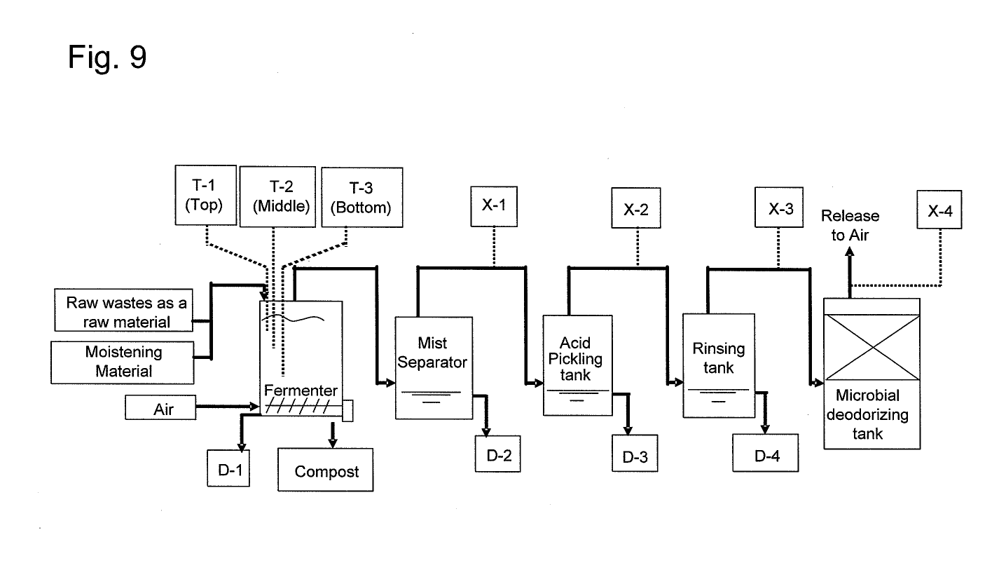

Using a fermentation apparatus corresponding to the fermentation apparatus 10 shown in FIG. 1 and having a general configuration shown in FIG. 9, a change in the temperature inside a fermentation tank when fermentation was carried out for 30 days was observed. In order to observe the deodorizing condition of the odor of gases produced at the time of fermentation in detail, a deodorizing device included a mist separator, an acid pickling tank, a rinsing tank and a microbial deodorizing tank.

Only one fermentation tank is shown in FIG. 9, and the other fermentation tanks are omitted in the figure.

### (1) Preparation of Microbial Deodorizing Tank

4pgm6 (Biotechnology center of National Institute of Technology and Evaluation, deposit number: FERM P-17867), 4pg2 (Biotechnology center of National Institute of Technology and Evaluation, deposit number: FERM P-17869), 4pg4 (Biotechnology center of National Institute of Technology and Evaluation, deposit number: FERM P-17870), 4pgm2 (Biotechnology center of National Institute of Technology and Evaluation, deposit number: FERM P-17865), 4pgm3 (Biotechnology center of National Institute of Technology and Evaluation, deposit number FERM P-17866), 1pg2 (Biotechnology center of National Institute of Technology and Evaluation, deposit number: FERM P-17868), and 4pgm8 (Biotechnology center of National Institute of Technology and Evaluation, deposit number: FERM P-17871) were used for the microbial deodorizing tank.

One L of distilled water was put in a 2-L beaker, 7.2 g of Malt Extract (Cat. number: 218630, made by Japan Becton, Dickinson and Company), 18.0 g of Yeast Extract (Cat. number: 212750, made by Japan Becton, Dickinson and Company), 7.2 g of glucose (special grade, made by WAKO Pure Chemical Industries, Ltd.) were dissolved therein, and sodium hydroxide was added in order to obtain a liquid culture medium with pH of 7.3.

Next, distilled water was added to the liquid culture medium in order to perform mess-up up to 1.8 L, separated and poured into 1 L of culture bottle 300 mL by 300 mL, and sterilized through auto clave (120° C., 15 minutes). The above-explained bacterial secondary stock was inoculated one by one in the culture medium in each sterilized culture bottle within a clean bench, and cultured by a rotary shaker for one to two nights under a condition of 38° C. and 120 rpm, thereby obtaining a suspension of microorganisms.

Next, 20 g of KH₂PO₄ (made by WAKO Pure Chemical Industries, Ltd., special grade), 10 g of CaCl₂.2H₂O (KANTO Chemical Co., Ltd., special grade), 10 g of MgSO₄.7H₂O (WAKO Pure Chemical Industries, Ltd., special grade) and 2.5 g of CoCl₂.6H₂O were dissolved in 10 L of water in order to prepare a mineral liquid. The mineral liquid was stocked in a room temperature.

Peat moss was used as the immobilizer for the microbial deodorizing tank. First, a pack of commercially available peat moss (substantially 50 L, made in Canada) was taken out from the pack, flexed, spread, and sprayed with 1 L of the above-explained mineral liquid. Next, the above-explained culture liquid was turned upside down and mixed, and 200 to 300 mL of such liquid per a pack of peat moss was sprayed.

The immobilized layer prepared as explained above was filled in the container for the microbial deodorizing tank, thereby preparing the microbial deodorizing tank.

### (2) Temperature Change in Fermentation Tank

In FIG. 9, a thermometer T-1, a thermometer T-2, and a thermometer T-3 were set at respective locations at 500 mm from the ceiling of the fermentation tank with the height of 1600 mm, at 800 mm, and at 1100 mm. The blown amount of air was set to be 1 m³/minute.

A loaded amount (kg) of raw materials, a water contents (%), a temperature inside the fermentation tank (° C.), an external temperature (° C.), a temperature difference (° C.) between the external temperature and the average temperature inside the fermentation tank, a drained amount (mL), and an smell index are shown in tables 3 to 5.

TABLE 3

| | | Fermentation (days) | | | | | | | | | |
|---|---|---|---|---|---|---|---|---|---|---|---|
| | | 1 | 2 | 3 | 4 | 5 | 6 | 7 | 8 | 9 | 10 |
| Organic waste amount | Organic waste as a | 50 | 50 | 0 | 50 | 37 | 42 | 47 | 36 | 36 | 0 |
| loaded (kg) | raw material | | | | | | | | | | |
| | Moistening Material | 50 | 1.5 | 0 | 2 | 25 | 25 | 25 | 20 | 15 | 0 |
| | (tips) | | | | | | | | | | |
| | Water content (%) | 60 | 60 | 0 | 60 | 60 | 60 | 60 | 60 | 60 | 0 |
| Temp. in fermenter (° C.) | T-1 | 14 | 13 | 13 | 13 | 13 | 44 | 60 | 64 | 66 | 66 |
| | T-2 | 14 | 13 | 13 | 27 | 43 | 53 | 62 | 64 | 67 | 67 |
| | T-3 | 12 | 14 | 14 | 22 | 33 | 41 | 49 | 53 | 64 | 64 |
| | Average in | 14 | 13.3 | 13.3 | 20.6 | 29 | 46 | 57 | 60 | 66.6 | 66.6 |
| | fermenter | | | | | | | | | | |
| Outer temp. (° C.) | | | 12 | 12 | 13 | 12 | 15 | 14 | 15 | 17 | 17 |
| Temp. difference between inside and outside | | | 1.3 | 1.3 | 7 | 17 | 31 | 43 | 45 | 49 | 49 |
| of the fermenter (° C.) | | | | | | | | | | | |
| Drain volume (mL) | D1 | 0 | 0 | 0 | 0 | 0 | 0 | 0 | 0 | 0 | 0 |
| | D2 | 0 | 0 | 0 | 0 | 0 | 0 | 0 | 15 | 1400 | 0 |
| | D3 | 0 | 0 | 0 | 0 | 0 | 0 | 0 | 0 | 0 | 0 |
| | D4 | 0 | 0 | 0 | 0 | 0 | 0 | 0 | 0 | 0 | 0 |
| Smell index | X1 Lv | | | | | 505 | 704 | 877 | 778 | 943 | 943 |
| | X2 Lv | | | | | 625 | 643 | 856 | 807 | 859 | 859 |
| | X3 Lv | | | | | 575 | 898 | 883 | 1046 | 979 | 979 |
| | X4 Lv | | | | | 61 | 352 | 317 | 225 | 361 | 361 |

| | |
|---|---|
| Compost amount mixed upside-down (kg) | 45 |

TABLE 4

| | | Fermentation (days) | | | | | | | | | |
|---|---|---|---|---|---|---|---|---|---|---|---|
| | | 11 | 12 | 13 | 14 | 15 | 16 | 17 | 18 | 19 | 20 |
| Organic waste amount | Organic waste as a | 35 | 36 | 40 | 0 | 0 | 0 | 0 | 0 | 0 | 0 |
| loaded (kg) | raw material | | | | | | | | | | |
| | Moistening Material | 15 | 15 | 13 | 0 | 0 | 0 | 0 | 0 | 0 | 0 |
| | (tips) | | | | | | | | | | |
| | Water content (%) | 60 | 60 | 60 | 0 | 0 | 0 | 0 | 0 | 0 | 0 |
| Temp. in fermenter (° C.) | T-1 | 66 | 61 | 65 | 68 | 69 | 71 | 71 | 69 | 62 | 63 |
| | T-2 | 66 | 56 | 63 | 66 | 67 | 66 | 66 | 62 | 58 | 61 |
| | T-3 | 63 | 52 | 60 | 61 | 62 | 61 | 61 | 59 | 54 | 52 |
| | Average in | 65 | 56.3 | 60.6 | 65.3 | 66 | 66 | 66 | 62.6 | 58 | 58.6 |
| | fermenter | | | | | | | | | | |
| Outer temp. (° C.) | | 19 | 13 | 11 | 19 | 9 | 10 | 10 | 7 | 6 | 8 |
| Temp. difference between inside and outside | | 46 | 43.3 | 51.6 | 46 | 57 | 56 | 56 | 55.6 | 52 | 50.6 |
| of the fermenter (° C.) | | | | | | | | | | | |
| Drain volume (mL) | D1 | 0 | 0 | 0 | 0 | 0 | 0 | 0 | 0 | 0 | 0 |
| | D2 | 4500 | 4750 | 3850 | 4400 | 8000 | 7000 | 0 | 15000 | 5000 | 5000 |
| | D3 | 0 | 0 | 2500 | 0 | 200 | 0 | 0 | 5000 | 8000 | 7000 |
| | D4 | 0 | 0 | 130 | 0 | 0 | 0 | 0 | 620 | 1500 | 2000 |
| Smell index | X1 Lv | 1012 | 1094 | 1020 | 1112 | 876 | 921 | 921 | 495 | 440 | 441 |
| | X2 Lv | 714 | 715 | 783 | 1211 | 787 | 813 | 813 | 410 | 448 | 470 |
| | X3 Lv | 871 | 872 | 1071 | 904 | 932 | 945 | 945 | 602 | 586 | 602 |
| | X4 Lv | 371 | 326 | 337 | 364 | 302 | 372 | 372 | 181 | 204 | 155 |

| | | | | | | | | |
|---|---|---|---|---|---|---|---|---|
| Compost amount mixed upside-down (kg) | 45 | 28 | 45 | 15 | 15 | 15 | 15 | 15 |

TABLE 5

| | | Fermentation (days) | | | | | | | | | | |
|---|---|---|---|---|---|---|---|---|---|---|---|---|
| | | 21 | 22 | 23 | 24 | 25 | 26 | 27 | 28 | 29 | 30 | Total |
| Organic waste amount | Organic waste as | 0 | 0 | 0 | 0 | 0 | 0 | 0 | 0 | 0 | 0 | 459.0 |
| loaded (kg) | a raw material | | | | | | | | | | | |
| | Moistening Material | 0 | 0 | 0 | 0 | 0 | 0 | 0 | 0 | 0 | 0 | 206.5 |
| | (tips) | | | | | | | | | | | |
| | Water content (%) | 0 | 0 | 0 | 0 | 0 | 0 | 0 | 0 | 0 | 0 | |
| Temp. in fermenter (° C.) | T-1 | 63 | 63 | 57 | 57 | 58 | 57 | 53 | 54 | 58 | 57 | |
| | T-2 | 63 | 63 | 58 | 58 | 59 | 59 | 50 | 50 | 49 | 46 | |
| | T-3 | 52 | 54 | 54 | 54 | 57 | 57 | 48 | 48 | 41 | 33 | |
| | Average in | 59.3 | 60 | 56.3 | 56.3 | 58 | 57.6 | 50.3 | 50.3 | 49.3 | 45.3 | |
| | fermenter | | | | | | | | | | | |
| Outer temp. (° C.) | | 7 | 8 | 7 | 7 | 8 | 7 | 12 | 12 | 12 | 12 | |
| Temp. difference between inside and outside | | 52.3 | 52 | 49.3 | 49.3 | 50 | 50.6 | 38.3 | 38.3 | 37.3 | 33.3 | |
| of the fermenter (° C.) | | | | | | | | | | | | |
| Drain volume (mL) | D1 | 0 | 0 | 0 | 0 | 0 | 0 | 0 | 0 | 0 | 0 | 0.0 |
| | D2 | 4000 | 5500 | 0 | 0 | 11500 | 3000 | 3000 | 3000 | 5000 | 4000 | 97,915.0 |
| | D3 | 475 | 3350 | 0 | 0 | 75 | 0 | 3500 | 710 | 30 | 70 | 30,910.0 |
| | D4 | 1600 | 1550 | 0 | 0 | 360 | 800 | 2250 | 410 | 2000 | 0 | 13,220.0 |
| Smell index | X1 Lv | 416 | 575 | 417 | 417 | 404 | 464 | 463 | 521 | 440 | 370 | |
| | X2 Lv | 414 | 650 | 437 | 437 | 385 | 425 | 431 | 479 | 420 | 307 | |
| | X3 Lv | 622 | 783 | 571 | 571 | 486 | 516 | 495 | 537 | 370 | 341 | |
| | X4 Lv | 203 | 340 | 165 | 165 | 127 | 221 | 147 | 166 | 250 | 204 | |

| | | | | | | | | | |
|---|---|---|---|---|---|---|---|---|---|
| Compost amount mixed upside-down (kg) | 15 | 15 | 15 | 15 | 30 | 30 | 30 | 30 | 30 |

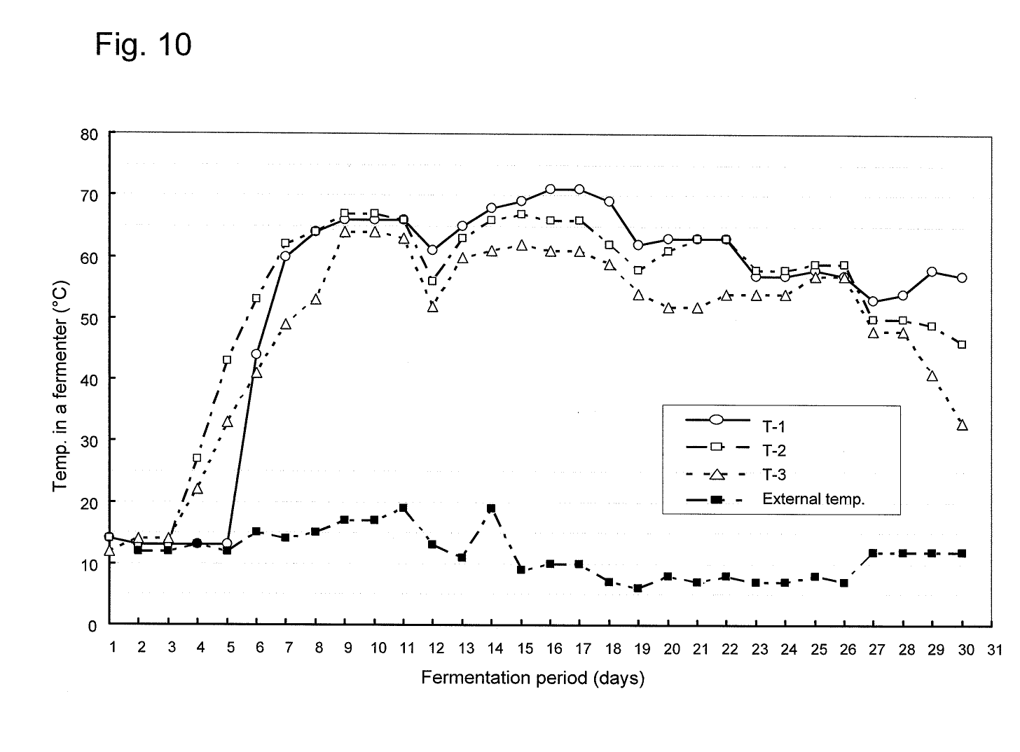

As shown in FIG. 10, the temperature inside the fermentation tank became equal to or higher than 49° C. at all of the thermometers after a week, and exceeded 60° C. at T-1 and T-2. T-1 exceeded 70° C. at 16th day, was equal to or higher than 60° C. up to 22nd day. Thereafter, the temperature dropped but showed 57° C. at 30th day.

Moreover, T-2 reached 67° C. at 15th day, dropped to 50° C. at 27th day, and became 46° C. at 30th day. T-3 reached 64° C. at 10th day, continuously became equal to or lower than 60° C. after 18th day, and dropped to 48° C. at 27th day. The temperature rapidly dropped to 41° C. at 29th day, and to 33° C. at 30th day.

### (3) Deodorizing Effect

How much the odor produced at the time of fermentation was deodorized was observed at four locations; (X-1) after gases evacuated from the evacuating opening of the fermentation tank passed through the mist separator; (X-2) after passed through the acid pickling tank; (X-3) after passed through the rinsing tank; and (X-4) after passed through the microbial deodorizing tank.

Measurement was carried out using odor sensors (XP329 made by New Cosmos Electronic Co., Ltd.). The results are shown in tables 3 to 5 and FIG. 11.

As shown in FIG. 11 and tables 3 to 5, the smell index was substantially zero up to 4th day of the fermentation process, but rapidly increased thereafter. The smell index of gases with smell increased from 5th day, and reached 921 behind the mist separator at 17th day. After the gases passed through the acid pickling tank, there was a tendency that the smell index decreased slightly, but it was not a sufficient deodorizing effect, and the same is true of the gases passed through the rinsing tank.

Conversely, after the gases passed through the microbial deodorizing tank prepared as explained above, the smell index remarkably decreased, so that it was demonstrated that the deodorizing effect by the microbial deodorizing tank 64 was high.

### Second Example

### Production of Composts from Organic Wastes

### (1) Material

Chips obtained by cutting sawdust, forest thinning and pruned branches of timber into small pieces were used as the water adjusting materials. Moreover, meats, fish, vegetables, fruits, processed foods, and rice bran were used as organic wastes at respective amounts indicated in table 6 below (total amount for 10 days).

TABLE 6

| Materials threw in | Weight (kg) | Ratio (%) |
|---|---|---|
| Meat & fish | 476.9 | 19.9 |
| Vegetables & fruits | 1556.7 | 65.5 |
| Processed foods | 8.9 | 0.4 |
| Rice bran | 90.7 | 3.8 |
| Total of garbage | 2133.1 | 89.2 |
| Total including Moistening Material | 257.7 | 10.8 |
| Total | 2390.8 | 100.0 |

### (2) Fermentation Process

Substantially 80 kg of the above-explained organic wastes and moistening material were put in the primary fermentation tank of the useful product producing apparatus shown in FIG. 1 at the first day (Feb. 7, 2008) in order to form the organic waste layer, and air was blown into the fermentation tank by activating the air pump, thereby starting a fermentation process.

On the next day, substantially 80 kg of the organic wastes and moistening material were put on the processed layer through the same procedure. Processed layers were formed through the same procedure for 10 days with the same amount of organic wastes and moistening material, and the fermentation process was kept in progress while adjusting the blown air volume so that the respective thermometers indicate temperatures shown in table 7 below.

TABLE 7

| | Maximum | Minimum |
|---|---|---|
| Thermometer | temperature (° C.) | temperature (° C.) |
| T₁ | 70° C. | 60° C. |
| T₂ | 60° C. | 50° C. |
| T₃ | 50° C. | 40° C. |

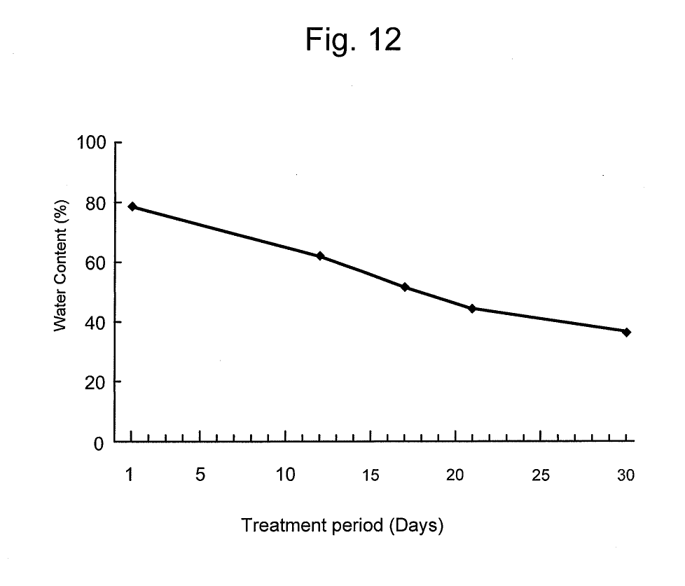

A change in the water contents of the organic wastes in the primary fermentation tank was observed. The result is shown in FIG. 12. As shown in FIG. 12, the water contents of the processed materials decreased from substantially 80% to substantially 40% during the fermentation process for 10 days in the primary fermentation tank.

Next, the primary processed materials were moved in the secondary fermentation tank 10S, water was supplied so that the water contents of the primary processed materials became substantially 60%, and a secondary process was carried out while adjusting the blown air volume so that the temperature range in the secondary fermentation tank 10S became as shown in table 8 below.

TABLE 8

| | Maximum | Minimum |
|---|---|---|
| Thermometer | temperature (° C.) | temperature (° C.) |
| T₁ | 60° C. | 40° C. |
| T₂ | 55° C. | 40° C. |
| T₃ | 50° C. | 40° C. |

Next, the secondary processed materials were moved in the third fermentation tank, water was supplied so that the water contents of the secondary processed materials became substantially 60%, and a third fermentation process was carried out while adjusting the blown air volume so that the temperature range in the third fermentation tank became as shown in table 9 below.

TABLE 9

| | Maximum | Minimum |
|---|---|---|
| Thermometer | temperature (° C.) | temperature (° C.) |
| T₁ | 50° C. | 40° C. |
| T₂ | 50° C. | 40° C. |
| T₃ | 40° C. | 40° C. |

After the process for 30 days completed, the processed layer RM₁ was taken out at 11th day by the ejecting device, and conveyed as the primary processed materials to the secondary fermentation tank by the convey device. After the primary processed materials loaded in the secondary fermentation tank formed a processed-material layer, water was supplementarily supplied by the water supply device under a condition that the water contents became 60%, and the air pump was activated in order to start a process. Thereafter, respective primary processed materials were successively taken out from the primary fermentation tank, and conveyed to the secondary fermentation tank.

The primary processed materials loaded in the secondary fermentation tank formed the layers in the order of loading-in, and respective layers were subjected to the secondary process.

After the secondary process for 10 days completed, respective processed layers were taken out by the ejecting device in a horizontal condition on the day 21, and conveyed as the secondary processed materials to the third fermentation tank by the convey device. After the primary processed materials loaded in the third fermentation tank formed the processed-material layers, water was supplementarily supplied by the water supply device under a condition that the water contents became 60%, and the air pump was activated in order to start a process. Thereafter, respective secondary processed materials were successively taken out from the secondary fermentation tank and conveyed to the third fermentation tank.

The secondary processed materials loaded in the third fermentation tank formed the layers in the order of loading-in, and respective layers were subjected to the third process.

### (3) Odor

Until the third process completed, ammonium odor, methyl disulfide and trimethylamine smell were observed using an odor sensor (XP329 made by New Cosmos Electronic Co., Ltd.).

The ammonium smell was 55 ppm at the entrance of the deodorizing tank, but was not detected at the exit of the deodorizing tank, and it was confirmed that equal to or larger than 99% of such smell was eliminated. Moreover, the methyl disulfide odor which was 320 ppm at the entrance of the deodorizing tank decreased at substantially half which was 140 ppm at the exit of the deodorizing tank. Likewise, trimethylamine odor which was 2.4 ppm decreased to 0.1 ppm which was equal to or smaller than 1/20 times. Through the foregoing observation, it was confirmed that the deodorizing device had a good deodorizing effect.

### (4) Temperature Change and Products, Etc., During Process

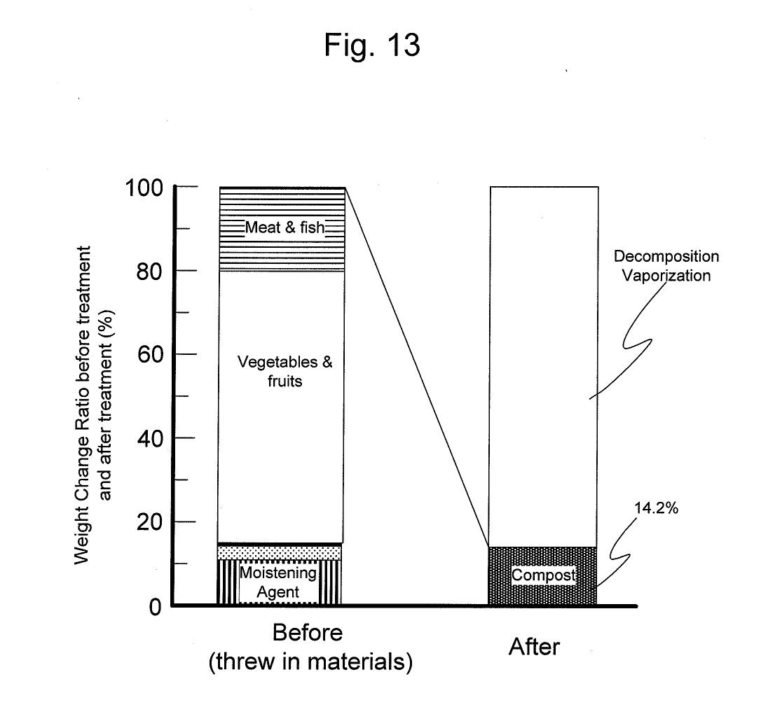

The third processed materials having undergone the process completed for 30 days were taken out as layers formed by such materials in a horizontal condition, and 340. 2 kg of products (composts) were obtained. The total amount of loaded materials and the amount of the products obtained are shown in table 10 below and FIG. 13.

TABLE 10

| Contents | Weight (kg) | Ratio (%) |
|---|---|---|
| Total amount threw in | 2390.8 | 100.0 |
| Products (compost) | 340.2 | 14.2 |
| Decomposition & vaporization amount | 2050.6 | 85.8 |

As is indicated above, the obtained products (composts) were substantially 14 wt % o the total amount of the loaded organic wastes and moistening material, and the amount of organic wastes remarkably decreased,

### (5) Analysis Results of Products (Composts)

The analysis results of the above-explained composts are shown in table 11 below. The preprocessing was carried out in accordance with the compost analyzing technique in the year of 1992. Moreover, the results are indicated by dry matter converted scores.

TABLE 11

| Subject to be analyzed | Result | unit | Analysis method | Lower limit |
|---|---|---|---|---|
| Total carbon | 45.7 | % | JIS M 8813 4.2 | |
| Ash | 6.5 | % | Fertilizer analysis method 3.2.1 Ignition ash method 1992 | 0.1 |
| Total Nitrogen (N) | 2.4 | % | Conform to Fertilizer analysis method 4.1.1.1 Sulfuric acid method 1992 | |
| Total Phosphorus (P₂O₅) | 1.4 | % | Fertilizer analysis method 4.2.3. E.a Vanadomolybdate ammonium method 1992 | |
| Total Potassium (K₂O) | 0.54 | % | Fertilizer analysis method 4.3.3 Flame Photometry 1992 | |
| Arsenic | 0.00017 | % | Fertilizer analysis method 5.24.2 Atomic Absorption Spectrometry 1992 | 0.000002 |
| Cadmium | <0.00001 | % | Fertilizer analysis method 5.6.1 Atomic Absorption Spectrometry 1992 | 0.00001 |
| Mercury | <0.000002 | % | Fertilizer analysis method 5.12.1 Reduced vaporization method 1992 | 0.000002 |
| Aqueous chlorine | 0.48 | % | Conform to Fertilizer analysis method 5.5.1 1992 | |
| Ammonia Nitrogen | 0.0066 | % | Fertilizer analysis method 4.1.2.1 Distillation method 1992 | |
| Organic Nitrogen | 2.4 | % | Conform to analysis method in Showa 53 and those mentioned above | |
| Water contents | 3.5 | % | Fertilizer analysis method 3.2.1 Heating loss method 1992 | 0.1 |
| Hydrogen ion concentration | 5.4(25.9° C.) | % | Fertilizer analysis method 3.3.1 Glass electrode method 1992 | |

As is indicated in the table 11, it was confirmed that composts containing nitrogen, phosphoric acid, and potash, all of which were in an appropriate amount were produced.

### Third Example

### Preparation of Feeding Stuffs from Organic Wastes

### (1) Material

A total amount of 1000 kg of palm shells with a water content rate of 15% were imported as organic wastes, the water contents was adjusted to 60% by supplying water, and such organic wastes were loaded in the fermentation tank. Through the same procedures as those of the first example other than the immobilizer of the deodorizing tank changed to the shells of palm and the outer skins thereof instead of peat moss, a fermentation process including primary fermentation for 10 days, secondary fermentation for 10 days, and third fermentation for 10 days, 30 days at total was carried out.

### (2) Preparation of Immobilizing Tank

Shells of palm and outer skins thereof were used as the immobilizer. Those were torn and cut into pieces with an appropriate size, and the mineral liquid and the suspension of the above-explained seven kinds of bacteria prepared through the same procedures as those of the first example were sprayed at the same rate. It was filled in the column and used as the microbial deodorizing tank.

### (3) Fermentation Process

The above-explained organic wastes were put in the primary fermentation tank through the same procedure as that of the first example, the layer was formed day by day, and a fermentation process for 10 days was carried out while controlling the supply volume of air so that respective thermometers became the temperature ranges shown in the table 7.

Next, the primary processed materials were moved in the secondary fermentation tank, and a secondary process was carried out so that respective thermometers became the temperature ranges shown in the table 8 through the same procedures as those of the first example. The water content of the primary processed materials was adjusted like the first example.

Thereafter, the secondary processed materials were moved in the third fermentation tank, and a third process was carried out so that respective thermometers became the temperature ranges shown in the table 9 through the same procedures as those of the first example. The water content of the secondary processed materials was adjusted like the first example.

### (4) Analysis Results of Products (Feeding Stuffs)

The results of chemical test (food constituent analysis) of second sample in accordance with a laboratory method are shown in table 12 below.

TABLE 12

| Subject to be analyzed | Contents (g/100 g except*¹) |
|---|---|
| Crude protein | 15.91 |
| Crude fat | 7.45 |
| Hydrocarbons | 67.71 |
| Water | 2.87 |
| Ash | 6.06 |
| Energy | 402*¹ |

*¹kcal/100 g

As is indicated in the table 12, crude protein and crude fat in addition to carbohydrate are contained, the energy amount is large, and the second sample can be used as feeding stuffs.

### Fourth Example

### Preparation of Fuel from Organic Wastes

The calorific value of the products produced through the same procedures as those of the second example was observed in accordance with HS K-2279. An automatic bomb calorimeter CP-4PJ (made by SHIMADZU Corporation) was used for observation.

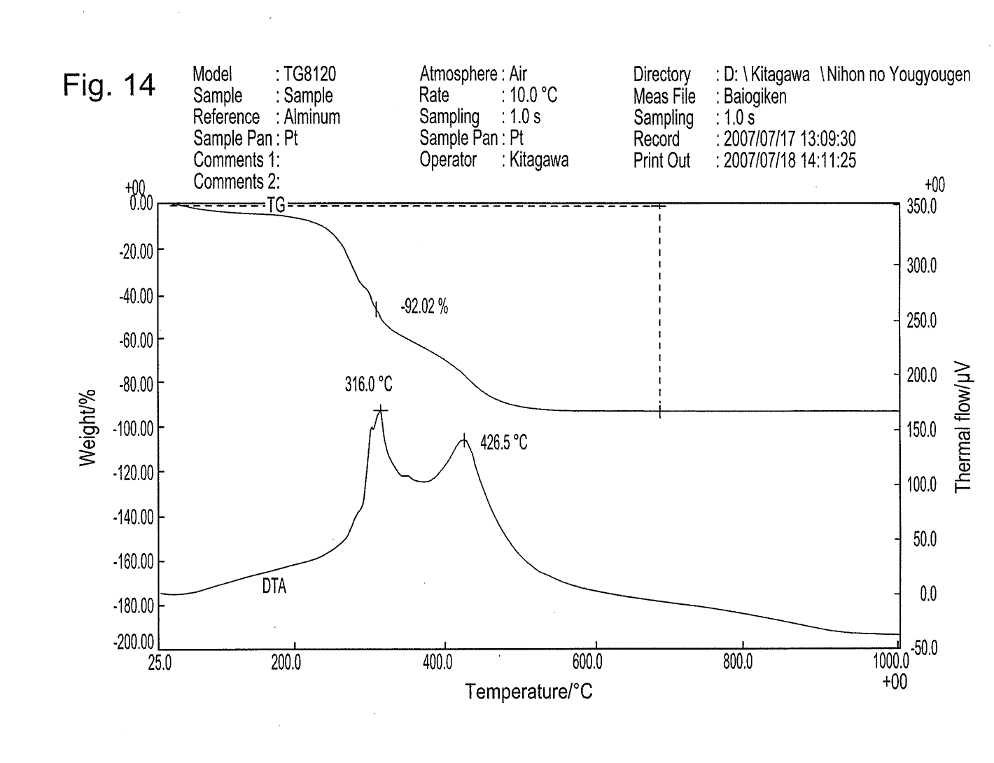

The calorific value of the processed materials (10.56 mg) derived from the shells of palm was observed, and a ceramic test (thermal analysis) at a temperature equal to or lower than 1000° C. was also carried out. A high-temperature type differential thermal analyzer TG 8120 (made by RIGAKU Corporation) was used for thermal analysis. Respective observation conditions and results are shown in table 13 and FIG. 14.

TABLE 13

| Measured item | Results | Method used | Apparatus used |
|---|---|---|---|
| Calorific power | 19,000 J/kg | Conform to JIS K-2279 | Shimadzu Corporation |
| | | | Auto-bomb calorimeter CP-4PJ |
| Thermal analysis (not | See FIG. 14 | Differential thermal analysis | Rigaku Corporation |
| higher than 1000° C.)) | | | High temperature type |
| | | | Differential thermal analyzer TG 8120 |

As is indicated in the table 13, the product produced with the shells of palm being as the organic waste raw materials has a high calorific value, and can be used as a fuel.

As shown in FIG. 13, from the thermal analysis result, peaks are observed around 316° C. and 426.5° C.

As is clear from the above-explained explanation, when the useful product producing apparatus of the present invention is used, it is possible to produce useful products, such as composts and feeding stuffs, from raw garbage and other organic wastes.

Because the fermentation tank has a deep depth, the surfacial area of the processed-material layer becomes small, so that air blown into each fermentation tank passes through the long flow path and reaches the upper portion of the fermentation tank. Hence, the time while the air is contacting the organic wastes or the processed materials becomes long, and the aerobic environment is efficiently maintained. Accordingly, the volume of air necessary for the fermentation process becomes equal to or smaller than ⅕ in comparison with conventional apparatuses. Moreover, the weight of the produced useful products becomes 15% or so of the loaded-in raw materials decomposed by microorganisms. Accordingly, the apparatus can be compact in size, and needs no heating from the exterior, which enables the process of organic wastes while accomplishing space saving and energy saving.

Moreover, according to the useful product processing apparatus of the present invention, the thermoduric bacteria which can grow in the fermentation tank gradually executes the fermentation process, so that ripened composts can be produced in a case in which composts are produced. The produced composts are processed for a long time under a high-temperature condition, so that the sterilization effect is high and most viruses are seemed to be killed. Accordingly, when using the composts, it is not necessary to make the composts re-fermented, and the high-quality composts which can be used efficiently can be produced.

The deodorizing device of the useful product producing apparatus of the present invention can efficiently deodorize the gases produced when the useful products are produced from the organic wastes, which expands the degree of freedom for installation of the apparatus at various locations.

The useful product producing apparatus of the present invention and the composts, feeding stuffs, fuels, etc., obtained from various organic wastes by the useful product producing apparatus are useful in the field of agriculture and in the environmental field.

1-21. (canceled)

22. A useful product producing apparatus which is a decomposing apparatus of organic wastes and comprises:
a fermentation device including a primary fermentation tank and a secondary fermentation tank and for decomposing the organic wastes;
an air supply device that forcibly supplies air to each of the primary fermentation tank and the secondary fermentation tank;
a deodorizing device that deodorizes gases evacuated from the primary fermentation tank and the secondary fermentation tank; and
a control device that controls the air supply device in order to control a fermentation process in the fermentation device, wherein
the useful product producing apparatus further comprises first convey means for conveying, to a second loading opening provided in an upper portion of the secondary fermentation tank, primary processed materials that are ejected from a first ejecting opening provided at a bottom portion of the primary fermentation tank after the organic wastes loaded in a first loading opening provided in an upper portion of the primary fermentation tank are processed in the primary fermentation tank,
the primary fermentation tank includes a first ejecting device which ejects a first lowermost layer from the first ejecting opening as the primary processed materials and which moves first stacked materials having the first lowermost layer eliminated therefrom downwardly in a horizontal condition after a process time of the first lowermost layer that is the lowermost layer in the first stacked materials has elapsed by a predetermined first process time in the primary fermentation tank, the first stacked materials including a plurality of substantially horizontal layers stacked together, the plurality of layers being successively formed loading period by loading period at which the organic wastes are loaded in the first loading opening through a daily routine, and the plurality of layers having different progress levels of decomposing carried out by a thermoduric bacteria flora under an aerobic condition in which a first air supply volume to the primary fermentation tank is controlled by the control device,
the secondary fermentation tank includes a second ejecting device which ejects a second lowermost layer from a second ejecting opening provided in a bottom portion of the secondary fermentation tank as the secondary processed materials and which moves second stacked materials having the second lowermost layer eliminated therefrom downwardly in a horizontal condition after a process time of the second lowermost layer that is the lowermost layer in the second stacked materials has elapsed by a predetermined second process time in the secondary fermentation tank, the second stacked materials including a plurality of substantially horizontal layers stacked together, the plurality of layers being successively formed loading period by loading period through the second loading opening, and the plurality of layers having partially different progress levels of decomposing carried out by another thermoduric bacteria flora under an aerobic condition in which a second air supply volume to the secondary fermentation tank is controlled by the control device, and
the deodorizing device includes:
an acid pickling tank that performs acid pickling on evacuated gases from the primary fermentation tank and the secondary fermentation tank;
a rinsing tank that performs rinsing on the gases having undergone acid pickling; and
a microbial deodorizing tank that performs microorganism-deodorizing on the gases having undergone rinsing.

23. The useful product producing apparatus according to claim 22, wherein
the first ejecting device includes a plurality of screw members each having a continuous spiral blades having a spiral pitch becoming narrow in a horizontal direction directed to the first ejecting opening, the plurality of screw members being arranged so as to be parallel to a bottom face of the primary fermentation tank and to have respective one ends protruding to an exterior of the primary fermentation tank from the first ejecting opening, and
the second ejecting device includes a plurality of screw members each having a continuous spiral blades having a spiral pitch becoming narrow in a horizontal direction directed to the second ejecting opening, the plurality of screw members being arranged so as to be parallel to a bottom face of the secondary fermentation tank and to have respective one ends protruding to an exterior of the secondary fermentation tank from the second ejecting opening.

24. The useful product producing apparatus according to claim 22 or 23, further comprising a water supply device that sprinkles water to the second stacked materials depending on a water containing amount of the second stacked materials.

25. The useful product producing apparatus according to claim 22 or 23, wherein a temperature inside the fermentation device is set to satisfy a condition in which a temperature produced by the thermoduric bacteria flora decomposing the organic wastes in the primary fermentation tank is higher than a temperature produced by the another thermoduric bacteria flora decomposing the primary processed materials in the secondary fermentation tank.

26. The useful product producing apparatus according to claim 22 or 23, wherein a moistening agent and/or the useful product is loaded in the primary fermentation tank together with the organic wastes.

27. The useful product producing apparatus according to claim 22 or 23, wherein the organic wastes are selected from the group consisting of a fecal substance of animal; a waste produced through breaking-up of animal body; a waste produced when green things are cooked; a waste produced through a process of obtaining an effective ingredient from green things; and a waste produced through a process for environment preservation, and mixture thereof.

28. The useful product producing apparatus according to claim 27, wherein the waste produced through breaking-up of an animal body includes various tissues and bones of animal.

29. The useful product producing apparatus according to claim 27, wherein the waste produced through a process of obtaining an effective ingredient from green things includes various tissues of green things.

30. The useful product producing apparatus according to claim 28, wherein the waste produced through a process of obtaining an effective ingredient from green things is selected from the group consisting of leaves, outer skins, and outer skin fibers of palm; shells and empty clusters of fruit; roots; and stocks.

31. The useful product producing apparatus according to claim 22 or 23, wherein
the thermoduric bacteria flora comprises a plurality of bacteria which suitably grow in a temperature range from 50 to 90° C., and which partially decompose the organic wastes to produce primary processed materials, and
the another thermoduric bacteria flora comprises a plurality of bacteria which suitably grow in a temperature range from 40 to 70° C., and which decompose the primary processed materials to produce secondary processed materials.

32. The useful product producing apparatus according to claim 22 or 23, wherein the microbial deodorizing tank includes a plurality of layers each of which includes microorganism immobilized to peat moss, outer skin fibers of timber, outer shell fibers of seed of timber, empty clusters of seed, or a porous ceramic substrate.

33. The useful product producing apparatus according to claim 32, wherein the microorganism immobilized to the plurality of layers is at least one kind of microorganisms selected from the group consisting of 4pgm6 (Biotechnology center of National Institute of Technology and Evaluation deposit number: FERM P-17867); 4pg2 (Biotechnology center of National Institute of Technology and Evaluation deposit number: FERM P-17869); 4pg4 (Biotechnology center of National Institute of Technology and Evaluation deposit number: FERM P-17870); 4pgm2 (Biotechnology center of National Institute of Technology and Evaluation deposit number: FERM P-17865); 4pgm3 (Biotechnology center of National Institute of Technology and Evaluation deposit number FERM P-17866); 1pg2 (Biotechnology center of National Institute of Technology and Evaluation deposit number: FERM P-17868); and 4pgm8 (Biotechnology center of National Institute of Technology and Evaluation deposit number: FERM P-17871).

34. The useful product producing apparatus according to any one of claims 22 to 33, wherein the useful product is selected from the group consisting of: fertilizer; feeding stuff; and fuel.

35. A useful product producing method comprising:
a primary process step of causing organic waste layers to be decomposed by a thermoduric bacteria flora under an aerobic condition in order to produce primary processed materials while moving the organic waste layers downwardly in a horizontal condition, the organic waste layers including substantially horizontal layers successively formed loading period by, loading period at which organic wastes are regularly loaded in a first loading opening provided in an upper portion of a primary fermentation tank;
a first take-out step of taking out a lowermost layer of the primary processed materials from a first ejecting opening provided in a bottom portion of the primary fermentation tank;
a convey step of conveying the primary processed materials taken out from the primary fermentation tank to a second loading opening provided in an upper portion of the secondary fermentation tank;
a secondary process step of causing the organic wastes not decomposed in the primary process step to be decomposed by another bacteria flora under an aerobic condition while moving secondary process layers downwardly in a horizontal condition, the secondary process layers including substantially horizontal layers stacked together and formed successively loading period by loading period at which the primary processed materials taken out in the first take-out step are loaded into the upper portion of the secondary fermentation tank through the convey step; and
a second take-out step of taking out a lowermost layer of the secondary process layers from an ejecting opening provided in a bottom portion of the secondary fermentation tank.

36. The useful product producing method according to claim 35, wherein the thermoduric bacteria flora comprises a plurality of bacteria which suitably grow in a temperature range from 40 to 90° C., and which partially decompose the organic wastes to produce primary processed materials, and
the another thermoduric bacteria flora comprises a plurality of bacteria which suitably grow in a temperature range from 40 to 70° C., and which decompose the primary processed materials to produce secondary processed materials.

37. The useful product producing method according to claim 35 or 36, wherein in the secondary process step, water is sprinkled to the secondary processed materials depending on a water containing amount of the secondary processed materials.
